(19) United States
(12) Patent Application Publication
(10) Pub. No.: US 2009/0177116 A1
(43) Pub. Date: Jul. 9, 2009

(54) BODY FLUID SAMPLING DEVICE AND BODY FLUID MEASURING DEVICE USING THE SAME

(75) Inventor: Takahiro Nakaminami, Osaka (JP)

Correspondence Address: MCDERMOTT WILL & EMERY LLP, 600 13TH STREET, NW, WASHINGTON, DC, 20005-3096, US

(73) Assignee: Body Fluid Sampling Device Fluid Measuring Device Using The Same

(21) Appl. No.: 12/296,596

(22) PCT Filed: Apr. 19, 2007
(86) PCT No.: PCT/JP2007/058533

§ 371 Date: Oct. 9, 2008

(30) Foreign Application Priority Data
Apr. 19, 2006 (JP) 2006-115761

(51) Int. Cl. A61B 5/00; G01N 21/85

(52) U.S. Cl. 600/573; 250/577

(57) ABSTRACT

A body fluid sampling device (100) includes: a connecting portion (105) to which a body fluid container is connected; a suctioning portion (117) which suctions a gas in the body fluid container connected to the connecting portion, to introduce a body fluid into the body fluid container; a suctioning passage (105a) which causes an inside of the suctioning portion and an inside of the body fluid container connected to the connecting portion, to be communicated with each other; a first electrode (112) which is disposed to extend across the suctioning passage and has gas permeability; a second electrode (113) which is disposed to extend across the suctioning passage and be opposed to the first electrode, and has gas permeability; and a detector which detects an electric signal generated between the first electrode and the second electrode. With this, the present invention provides the body fluid sampling device capable of detecting improper suctioning of the body fluid, and a body fluid measuring device using the body fluid sampling device.

### TECHNICAL FIELD

The present invention relates to a body fluid sampling device which is used to obtain a body fluid, and a body fluid measuring device which uses the body fluid sampling device to measure the amount of a specific substance contained in the obtained body fluid.

### BACKGROUND ART

A particular method for obtaining a certain amount of the body fluid is used to, for example, test, analyze, and quantitate the specific substance contained in the body fluid.

One example of such method for obtaining a certain amount of the body fluid is pipetting using a pipetter. In the pipetting of the body fluid using the pipetter, an operation of obtaining the body fluid is comparatively easy. Therefore, the pipetting of the body fluid using the pipetter has been commonly used by many analysis engineers and the like. The pipetter is one type of liquid sampling device which accurately measures a certain amount of liquid when testing, analyzing, or quantitating a substance. Using this pipet, the analysis engineers and the like can accurately obtain liquids, such as the body fluid.

In the case of using the liquid sampling device, such as the pipet, to prevent an obtained second liquid from being contaminated by a first liquid, the analysis engineers and the like are required to obtain the second liquid after the first liquid has been obtained and at least a portion of the liquid sampling device which has contacted the first liquid has been cleaned. To be specific, in the case of using conventional liquid sampling devices, it is necessary to clean, after each use, the portion which has contacted the liquid. This has been a cause of deteriorating work efficiency and test accuracy.

To solve such problem, a liquid sampling device has been proposed, in which a detachable sampling container is attached to a sampling device including a suction mechanism, and air of the same volume as the amount of the body fluid to be obtained is suctioned from the sampling container, thereby obtaining the body fluid in the sampling container through a liquid supply port of the sampling container (Patent Document 1 for example).

In accordance with the configuration of the proposed liquid sampling device, since the sampling container for obtaining the body fluid can be discarded after use, it is unnecessary to clean, after each use, the portion which has contacted the body fluid. To be specific, in accordance with the configuration of the liquid sampling device, since the operation of obtaining the body fluid is comparatively easy, and the cause of deteriorating the work efficiency and the test accuracy is removed, it is possible to, for example, preferably test, analyze, and quantitate the specific substance contained in the body fluid. Therefore, the configuration of the proposed liquid sampling device is most commonly used at present.

Patent Document 1: Japanese Laid-Open Patent Application Publication 6-109603

### DISCLOSURE OF THE INVENTION

### Problems to be Solved by the Invention

In the case of using the above conventional liquid sampling device, during the operation of obtaining the body fluid, the analysis engineers and the like are required to place at all times the liquid supply port of the sampling container below a fluid level of the body fluid to be obtained.

However, since the suction mechanism keeps on suctioning even if the liquid supply port of the sampling container moves to be located, for even a moment during the operation of obtaining the body fluid, above the fluid level of the body fluid to be obtained, the body fluid splashes into the suction mechanism of the liquid sampling device through the sampling container. That is, in the case of using the conventional liquid sampling device, the inside of the suction mechanism is contaminated by the body fluid in some cases.

Moreover, in the case of using the conventional liquid sampling device, even if the body fluid has splashed into the suction mechanism of the liquid sampling device through the sampling container, the splashing of the body fluid cannot be visually checked. That is, in the case of using the conventional liquid sampling device, the analysis engineers and the like cannot recognize the contamination of the liquid sampling device caused by improper suctioning of the body fluid.

The present invention was made to solve the above conventional problems, and the first object of the present invention is to provide a body fluid sampling device which can prevent the body fluid from being improperly suctioned into the body fluid sampling device, and which even if the body fluid is improperly suctioned, can surely detect the improper suctioning of the body fluid.

Moreover, the second object of the present invention is to provide a preferable body fluid measuring device which includes the body fluid sampling device having the above first object and excels in operability and convenience. Means for Solving the Problems

To solve the above conventional problems, a body fluid sampling device according to the present invention includes: a connecting portion to which a body fluid container is connected; a suctioning portion which suctions a gas in the body fluid container connected to the connecting portion, to introduce a body fluid into the body fluid container; a suctioning passage which causes an inside of the suctioning portion and an inside of the body fluid container connected to the connecting portion, to be communicated with each other; a first electrode which is disposed to extend across the suctioning passage and has gas permeability; a second electrode which is disposed to extend across the suctioning passage and be opposed to the first electrode, and has gas permeability; and a detector which detects an electric signal generated between the first electrode and the second electrode.

With this configuration, the body fluid sampling device includes the first electrode which is disposed to extend across the suctioning passage and has the gas permeability, the second electrode opposed to the first electrode, and the detector which detects the electric signal generated between the first electrode and the second electrode. Therefore, in a case where the body fluid has been improperly suctioned into the body fluid sampling device, and the body fluid has reached between the first electrode and the second electrode, the detector can detect the electric signal generated between these two electrodes. Thus, it is possible to detect that the body fluid has been improperly suctioned into the body fluid sampling device.

In this case, the body fluid sampling device further includes a separator which is disposed between the first electrode and the second electrode and is able to be impregnated with the body fluid.

With this configuration, since the body fluid sampling device further includes the separator which is disposed between the first electrode and the second electrode and is able to be impregnated with the body fluid, the separator disposed between the first electrode and the second electrode is surely impregnated with the body fluid having been improperly suctioned into the suctioning passage of the body fluid sampling device. Thus, since the suctioned body fluid surely contacts these two electrodes, it is possible to further highly precisely detect improper suctioning of the body fluid.

Moreover, in the above case, the body fluid sampling device further includes a filter which is disposed inside the suctioning passage and is able to suppress an intrusion of the body fluid.

With this configuration, since the body fluid sampling device further includes the filter which is disposed inside the suctioning passage and is able to suppress the intrusion of the body fluid, the filter surely captures the droplets of the body fluid, and it is possible to surely prevent the body fluid from getting into the suction mechanism. With this, it is possible to surely detect that the body fluid, whose amount exceeds an amount of liquid which can be captured by the filter, has been improperly suctioned.

In this case, in the body fluid sampling device, the filter is configured to contact at least one of the first electrode and the second electrode.

With this configuration, by causing the filter to contact at least one of the first electrode and the second electrode, it is possible to further surely prevent the intrusion of the body fluid, and also possible to further preferably hold the body fluid having been introduced between the first electrode and the second electrode. Moreover, since the filter is impregnated with the body fluid when the filter is saturated with the body fluid droplets, the body fluid captured by the filter easily contacts two electrodes. Therefore, it is possible to further surely detect the existence of the body fluid droplets.

Moreover, in the above case, the body fluid sampling device further includes a surrounding member which integrally surrounds the first electrode and the second electrode.

With this configuration, since the first electrode and the second electrode can be handled as one integrated component, the number of components can be reduced, and the configuration of the body fluid sampling device can be simplified.

Meanwhile, in the above case, the body fluid sampling device further includes a control portion which causes, based on the electric signal detected by the detector, the suctioning portion to stop suctioning of the gas.

With this configuration, the body fluid sampling device further includes the control portion which causes, based on the electric signal detected by the detector, the suctioning portion to stop suctioning of the gas. Therefore, in a case where the body fluid is improperly suctioned, it is possible to immediately stop the suctioning of the suctioning portion. To be specific, since the suctioning of the body fluid stops when detecting the body fluid by the detector, it is possible to surely prevent the further intrusion of the body fluid into the suctioning portion.

Moreover, a first body fluid measuring device according to the present invention includes: any one of the above body fluid sampling devices; a light source which irradiates with light the body fluid introduced into the body fluid container connected to the connecting portion; a photoreceiver which receives the light emitted from the body fluid container irradiated by the light source; and a calculating unit which is used to measure the amount of a specific substance in the body fluid based on the light received by the photoreceiver.

With this configuration, since the body fluid measuring device includes the characteristic body fluid sampling device according to the present invention, it is possible to provide the preferable body fluid measuring device which excels in operability and convenience. Moreover, it is possible to carry out an optical measurement of the body fluid while maintaining a state where the detachable body fluid container holds the body fluid.

Moreover, a second body fluid measuring device according to the present invention includes: any one of the above body fluid sampling devices; a pair of measuring electrodes disposed inside the body fluid container and a pair of connecting terminals which are electrically connectable to the pair of measuring electrodes; and a calculating unit which is used to detect an electric signal generated between the pair of measuring electrodes via the pair of connecting terminals, and measure the amount of a specific substance in the body fluid based on the detected electric signal.

Even with this configuration, since the body fluid measuring device includes the characteristic body fluid sampling device according to the present invention, it is possible to provide the preferable body fluid measuring device which excels in operability and convenience. Moreover, it is possible to carry out an electrochemical measurement while maintaining a state where the detachable body fluid container holds the body fluid.

### Effects of the Invention

The present invention is carried out in accordance with “Means for Solving the Problems” above, and has an effect of being capable of providing a body fluid sampling device which can prevent the body fluid from being improperly suctioned into the body fluid sampling device, and which even if the body fluid is improperly suctioned, can surely detect the improper suctioning of the body fluid.

Moreover, the present invention has an effect of being capable of providing a preferable body fluid measuring device which includes the above characteristic body fluid sampling device and excels in operability and convenience.

### BRIEF DESCRIPTION OF THE DRAWINGS

FIG. 1 is a perspective view schematically showing the configuration of an appearance of a body fluid sampling device according to Embodiment 1.

FIG. 2 is a cross-sectional view schematically showing the configuration of a cross section of the body fluid sampling device.

FIG. 3 is a block diagram schematically showing an internal configuration of the body fluid sampling device.

FIG. 4 is a front view schematically showing a portion including a body fluid container and the vicinity of a connecting portion of the body fluid sampling device when the body fluid container is connected to the connecting portion.

FIG. 5 is a longitudinal sectional view schematically showing the portion including the body fluid container and the vicinity of the connecting portion of the body fluid sampling device when the body fluid container is connected to the connecting portion.

FIG. 6 is a side view schematically showing the portion including the body fluid container and the vicinity of the connecting portion of the body fluid sampling device when the body fluid container is connected to the connecting portion.

FIG. 7 is a cross-sectional view schematically showing the configuration of a cross section taken along line A-A′ of FIG. 5.

FIG. 8 is a cross-sectional view schematically showing the configuration of a cross section taken along line B-B′ of FIG. 5.

FIG. 9 is a cross-sectional view schematically showing the configuration of a cross section taken along line C-C′ of FIG. 5.

FIG. 10 is a cross-sectional view schematically showing the configuration of a cross section taken along line D-D′ of FIG. 5.

FIG. 11 is a cross-sectional view schematically showing the configuration of a cross section taken along line E-E′ of FIG. 5.

FIG. 12 is a perspective view schematically showing the configuration of an appearance of each of a first electrode and a second electrode included in the body fluid sampling device.

FIG. 13 is an enlarged perspective view schematically showing a void of each of the first electrode and the second electrode included in the body fluid sampling device.

FIG. 14 is a perspective view schematically showing the configuration of an appearance of a modification example of each of the first electrode and the second electrode included in the body fluid sampling device.

FIG. 15 is a longitudinal sectional view schematically showing the portion including the body fluid container and the vicinity of the connecting portion of the body fluid sampling device according to Embodiment 2 when the body fluid container is connected to the connecting portion.

FIG. 16 is a cross-sectional view schematically showing the configuration of a cross section taken along line G-G′ of FIG. 15.

FIG. 17 relates to a modification example of the body fluid sampling device, and is a longitudinal sectional view schematically showing the portion including the body fluid container and the vicinity of the connecting portion of the body fluid sampling device when the body fluid container is connected to the connecting portion.

FIG. 18 is a cross-sectional view schematically showing the configuration of a cross section taken along line I-I′ of FIG. 17.

FIG. 19 is a longitudinal sectional view schematically showing the portion including the body fluid container and the vicinity of the connecting portion of the body fluid sampling device according to Embodiment 3 when the body fluid container is connected to the connecting portion.

FIG. 20 is a cross-sectional view schematically showing the configuration of a cross section taken along line H-H′ of FIG. 19.

FIG. 21 are longitudinal sectional views schematically showing the configuration of major components of the body fluid sampling device according to Embodiment 4. FIG. 21(a) is a longitudinal sectional view schematically showing the arrangement of the first electrode, the second electrode, a separator, and a filter. FIG. 21(b) is a longitudinal sectional view schematically showing the arrangement of an assembly of the first electrode, the second electrode, the separator, and the filter.

FIG. 22 is a perspective view schematically showing the configuration of an appearance of the body fluid container used in Embodiment 5.

FIG. 23 is a cross-sectional view schematically showing the configuration of a cross section taken along line J-J′ of FIG. 22.

FIG. 24 is a cross-sectional view schematically showing the configuration of a cross section of the body fluid sampling device according to Embodiment 5.

FIG. 25 is a block diagram schematically showing an internal configuration of a body fluid measuring device.

FIG. 26 is a cross-sectional view schematically showing the configuration of a cross section of the body fluid measuring device according to Embodiment 6.

FIG. 27 is a longitudinal sectional view schematically showing the portion including the body fluid container and the vicinity of the connecting portion of the body fluid measuring device when the body fluid container is connected to the connecting portion.

FIG. 28 is a cross-sectional view schematically showing the configuration of a cross section taken along line K-K′ of FIG. 27.

FIG. 29 is a cross-sectional view schematically showing the configuration of a cross section taken along line L-L′ of FIG. 28.

FIG. 30 is a block diagram schematically showing an internal configuration of the body fluid measuring device.

FIG. 31 is a perspective view schematically showing the configuration of an appearance of a modification example of the body fluid container used in Embodiment 6.

### EXPLANATION OF REFERENCE NUMBERS

100 body fluid sampling device

101 body fluid container attaching portion

102 body fluid suction start button

103 body fluid discharge button

104 display portion

105 connecting portion

105a suctioning passage

106, 111 O-ring

107 cylinder

108 plunger

109 plunger joint

110 motor

112 first electrode

113 second electrode

114 separator

115 voltage applying portion

116 detector

117 suctioning portion

118 controller

118a calculating portion

120 first cylinder member

121 second cylinder member

122 first lead

123 second lead

124 cutout portion

130 casing

150 mesh electrode

200, 300, 500, 700 body fluid container

201, 302 space

202, 303, 704 opening

203, 307, 702 body fluid introducing port

301 base body

304 light incident portion

305 light emitting portion

306 reagent holding portion

308 first member

309 second member

400, 600 body fluid measuring device

401 light source

402 photoreceiver

403 timer portion

501, 710 first measuring electrode

501a first electrically-conductive portion

501b, 502b lead

501c, 502c cover

502, 712 second measuring electrode

502a second electrically-conductive portion

601 first connecting terminal

602 second connecting terminal

603 measuring voltage applying portion

604 electric signal measuring portion

605 third lead

606 fourth lead

701 metal fine wire

702 void

703 cover

706 hollow quadrangular prism portion

708 hollow quadrangular pyramid portion

800 electrode

801 through hole

901 filter

910 surrounding member

### BEST MODE FOR CARRYING OUT THE INVENTION

Hereinafter, the best mode for carrying out the present invention will be explained in detail in reference to the drawings.

### Embodiment 1

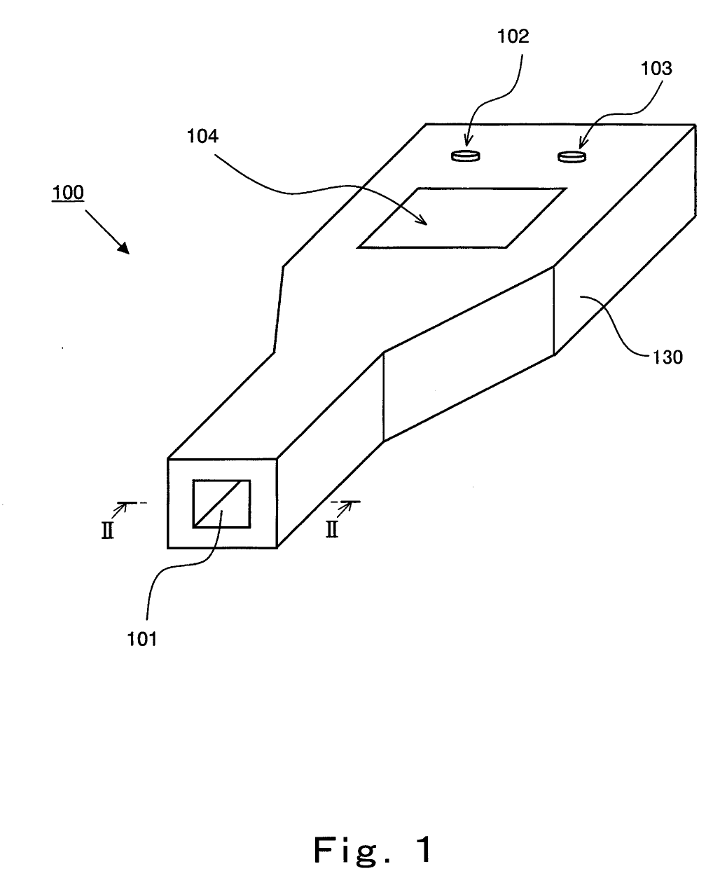

First, the configuration of a body fluid sampling device according to Embodiment 1 will be explained in reference to FIGS. 1 to 3.

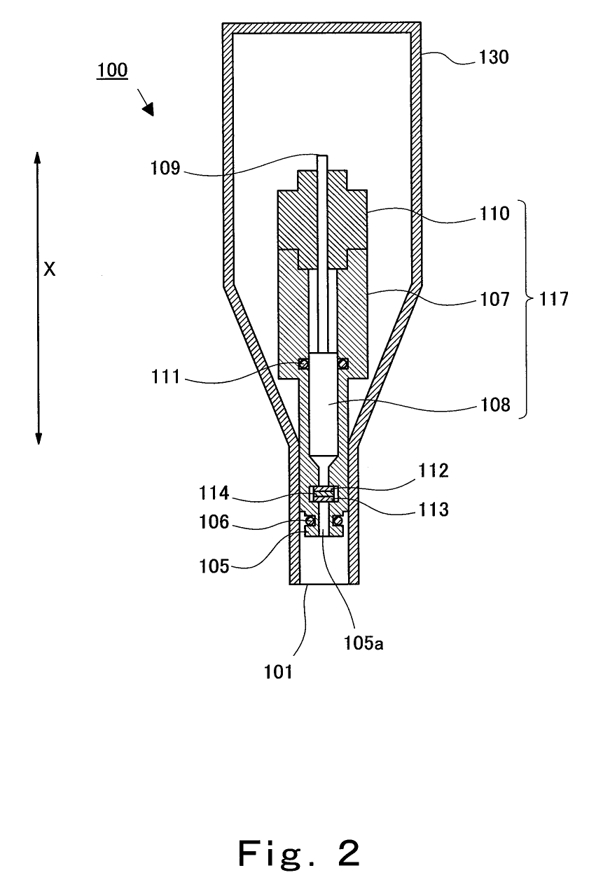

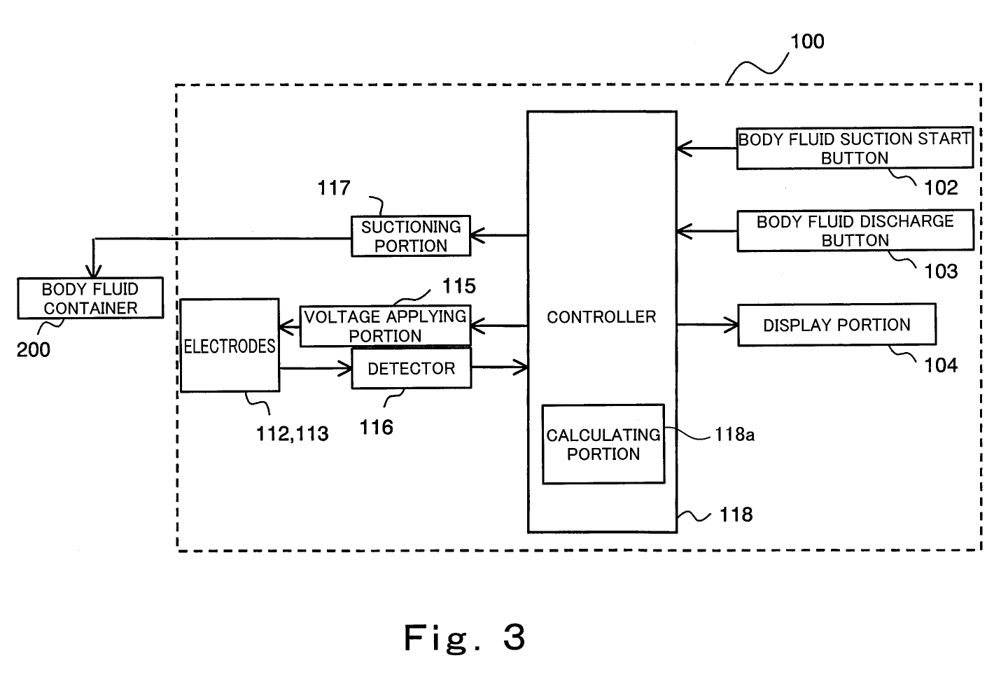

FIG. 1 is a perspective view schematically showing the configuration of an appearance of the body fluid sampling device according to Embodiment 1 of the present invention. FIG. 2 is a cross-sectional view schematically showing the configuration of a cross section of the body fluid sampling device. FIG. 3 is a block diagram schematically showing an internal configuration of the body fluid sampling device.

As shown in FIG. 1, a body fluid sampling device 100 according to the present embodiment includes a casing 130. The casing 130 includes: a body fluid container attaching portion 101 to which a body fluid container is detachably attached; a body fluid suction start button 102 by which the supply of a body fluid to the body fluid container starts; a body fluid discharge button 103 by which the body fluid in the body fluid container is discharged to a paper cup or the like; and a display portion 104, such as a liquid crystal display.

As shown in FIG. 2, a connecting portion 105 is disposed inside the body fluid container attaching portion 101. The body fluid container is attached to the connecting portion 105. Specifically, when attaching the body fluid container, the connecting portion 105 is inserted into a suction port of the body fluid container. To increase adhesion between the connecting portion 105 and the suction port of the body fluid container, an O-ring 106 made of rubber covered with fluorocarbon resin (Teflon (trademark) for example) is disposed around the connecting portion 105. With this, since air is prevented from leaking from the connecting portion 105 during suctioning, it is possible to further surely realize obtaining of the body fluid, which excels in quantitativity.

In the present embodiment, the O-ring 106 is fitted in a cutout portion formed on an outer surface of the connecting portion 105. In the present embodiment, an outer diameter of the cutout portion and an inner diameter of the O-ring 106 are set to be substantially equal to each other.

As above, to surely secure airtightness between the connecting portion 105 and the body fluid container connected to the connecting portion 105, it is preferable that the O-ring be disposed around the connecting portion 105. With this configuration, since the adhesion between the connecting portion 105 and the body fluid container increases dramatically, and the leakage of suctioned gas is suppressed effectively, the suctioning of the body fluid by a below-described suctioning portion 117 is satisfactorily carried out. In a case where the connecting portion 105 is made of resin, a circumferential protruding portion made of the same material may be formed on the connecting portion 105, thereby increasing the adhesion between the connecting portion 105 and the body fluid container. Moreover, a circumferential protruding portion made of the same material as the body fluid container may be formed on a contact surface of the body fluid container which contacts the connecting portion 105, thereby increasing the adhesion between the connecting portion 105 and the body fluid container. In these cases, the protruding portion may be constructed of a plurality of different protruding portions. Alternatively, highly viscous oil, such as grease, may be applied to the connecting portion, thereby increasing the adhesion between the connecting portion and the body fluid container. In this case, the use of the highly viscous oil may be combined with the use of the circumferential protruding portion.

In the case of utilizing the body fluid sampling device 100 according to the present embodiment, it is preferable that the body fluid container be detachably connected to the connecting portion 105 of the body fluid sampling device 100. With this configuration, the body fluid container can be replaced very easily. In addition, since the body fluid container is disposable, it becomes unnecessary to clean the body fluid container in the case of repeatedly carrying out the measurements.

As shown in FIG. 2, the body fluid sampling device 100 includes in the casing 130 a motor 110 which causes a plunger 108, disposed inside a cylinder 107, to move via a plunger joint 109. The motor 110, the plunger 108, and the cylinder 107 correspond to a suctioning portion which suctions the body fluid into the body fluid container. An O-ring 111 is disposed in the cylinder 107b to secure airtightness between the cylinder 107 and the plunger 108.

In the present embodiment, the suctioning portion 117 is configured such that the plunger 108 in the cylinder 107 is actuated by the motor 110, i.e., a linear stepping motor.

The stepping motor is such a motor that its rotating shaft rotates at a specific rotation angle for each input pulse signal. Since the rotation angle of the rotating shaft is determined in accordance with the number of pulses, the stepping motor does not require an encoder for positioning. That is, an operational distance of the plunger (piston) can be controlled in accordance with the number of input pulses. In the stepping motor, a rotational movement of the motor is converted to a desired translatory movement by using, for example, a gear mechanism, and a translatory mechanism in which an external thread and an internal thread are combined with each other. With this, the stepping motor causes the plunger 108 to move linearly. The linear stepping motor is configured such that the translatory mechanism in which the external thread and the internal thread are combined with each other is incorporated in the motor, and the plunger joint, i.e., a rod-like movable portion carries out the translatory movement in accordance with the number of input pulses. On this account, in the case of using the linear stepping motor, the plunger joint may be directly connected to the plunger, thereby simplifying the configuration of the body fluid sampling device 100.

In the present embodiment, the cylinder 107 and the connecting portion 105 are made of acrylonitrile butadiene styrene copolymer resin (ABS resin). As shown in FIG. 2, a suctioning passage 105a formed of a cylindrical space is formed in the cylinder 107 and the connecting portion 105 so as to penetrate through the connecting portion 105. The suctioning passage 105a extends in a direction substantially parallel to a direction (that is, a longitudinal direction of the body fluid sampling device 100) indicated by an arrow X of FIG. 2.

In the body fluid sampling device 100 according to the present embodiment, a first electrode 112 and a second electrode 113 are disposed to extend across the suctioning passage 105a, to sandwich a separator 114 capable of being impregnated with the body fluid, and to be opposed to each other. The first electrode 112 and the second electrode 113 are disposed to extend in a direction substantially perpendicular to the direction indicated by the arrow X of FIG. 2. An area of a main surface of each of the first electrode 112 and the second electrode 113 is larger than an area of a cross section (that is, a cutting surface substantially perpendicular to the direction indicated by the arrow X of FIG. 2) of the suctioning passage 105a.

In the present embodiment, it is preferable that the width (or the diameter or the area of the cross section) of at least one of the first electrode 112 and the second electrode 113 be larger than the width (or the diameter or the area of the cross section) of the suctioning passage 105a. With this configuration, since the cross section of the suctioning passage 105a is completely occupied by the electrode, it is possible to prevent the splashed body fluid from being adhered to a wall surface of the suctioning passage 105a without being captured by the electrode. In addition, since the splashed body fluid can be prevented from getting into the suctioning portion 105a, the existence of the splash can be further surely detected by the electrode. Although it is preferable that the width of at least one of the first electrode 112 and the second electrode 113 be larger than the width of the suctioning passage 105a as described above, the width of at least one of the first electrode 112 and the second electrode 113 may be not less than 60% and less than 100% of the with of the suctioning passage 105a.

In the body fluid sampling device 100, each of the first electrode 112 and the second electrode 113 has voids. These voids penetrate through each electrode. Since gas can move through these voids, the first electrode 112 and the second electrode 113 have gas permeability. This configuration reduces the degree of interference of movement of gas from the inside of the suctioning passage 105a to the suctioning portion 117 when suctioning the body fluid by the suctioning portion 117.

As shown in FIG. 3, the body fluid sampling device 100 includes: a voltage applying portion 115 which applies a voltage between the first electrode 112 and the second electrode 113; a detector 116 which detects an electric signal generated between the first electrode 112 and the second electrode 113; a controller 118 that is a control portion which controls, for example, the suctioning portion 117 which suctions the body fluid into a body fluid container 200. The suctioning portion 117 corresponds to the motor 110, the plunger 108, and the cylinder 107 shown in FIG. 2. The controller 118 is constituted by a microcomputer for example, and includes a calculating portion 118a. The calculating portion 118a executes predetermined calculation in the body fluid sampling device 100. An output voltage of the voltage applying portion 115 is generally about 0.2 V to 1 V, i.e., a comparatively low voltage, although it depends on the type of the body fluid. The detector 116 is a current sensor for example, and is configured to be able to detect a current of about several μA to several mA.

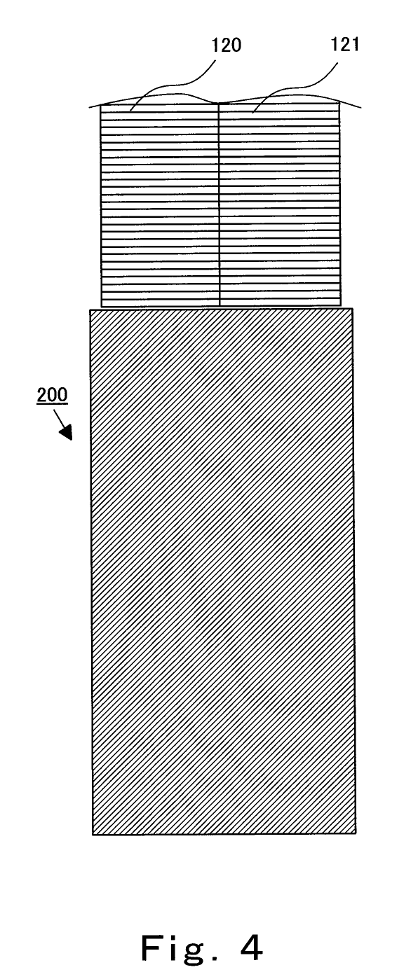

Next, a specific configuration of the vicinity of the connecting portion of the body fluid sampling device according to Embodiment 1 will be explained in detail in reference to FIGS. 4 to 12.

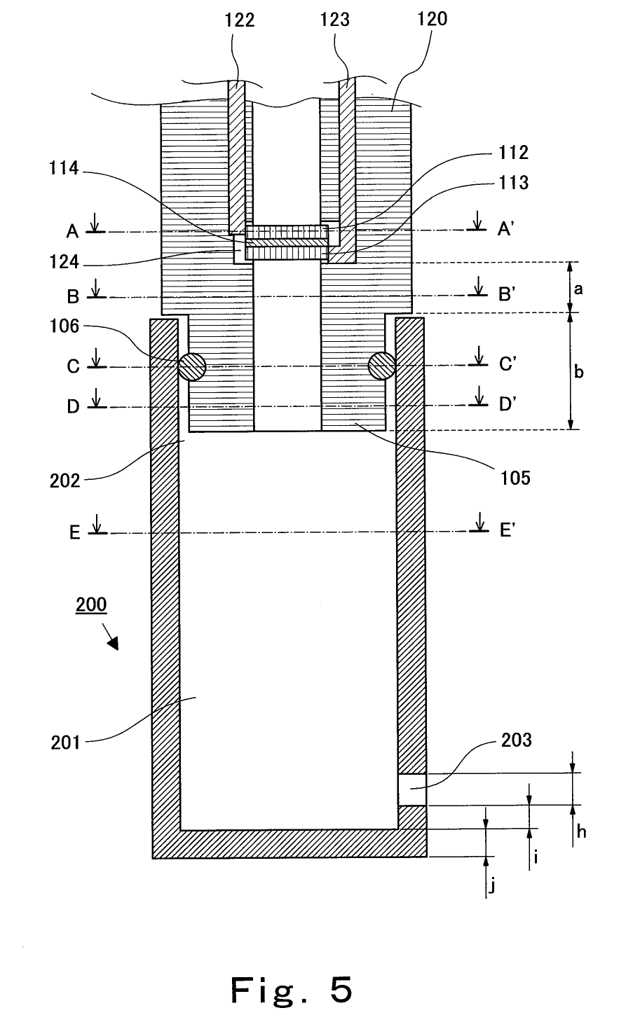

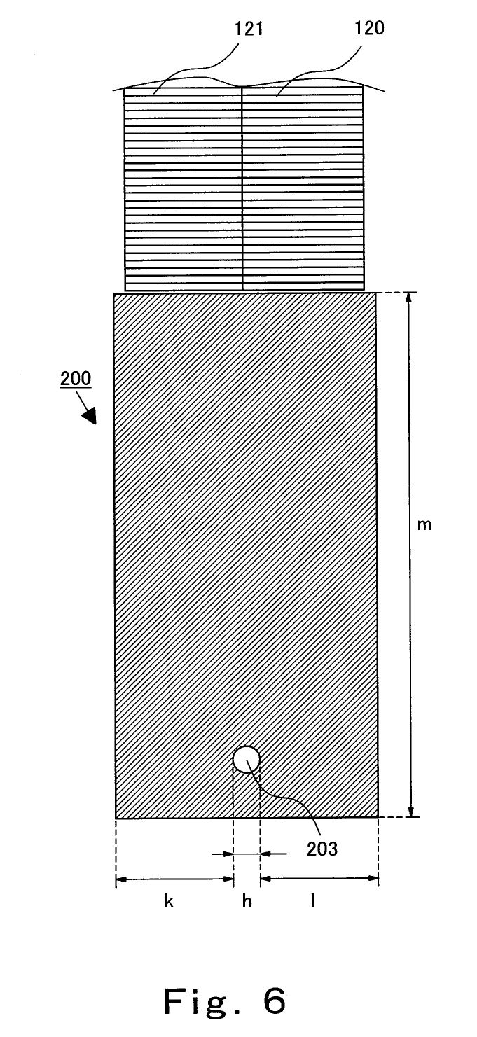

FIG. 4 is a front view schematically showing a portion including the body fluid container and the vicinity of the connecting portion of the body fluid sampling device when the body fluid container is connected to the connecting portion. FIG. 5 is a longitudinal sectional view schematically showing the portion including the body fluid container and the vicinity of the connecting portion of the body fluid sampling device when the body fluid container is connected to the connecting portion. FIG. 6 is a side view schematically showing the portion including the body fluid container and the vicinity of the connecting portion of the body fluid sampling device when the body fluid container is connected to the connecting portion. In FIGS. 4 to 6, the casing is omitted for convenience sake.

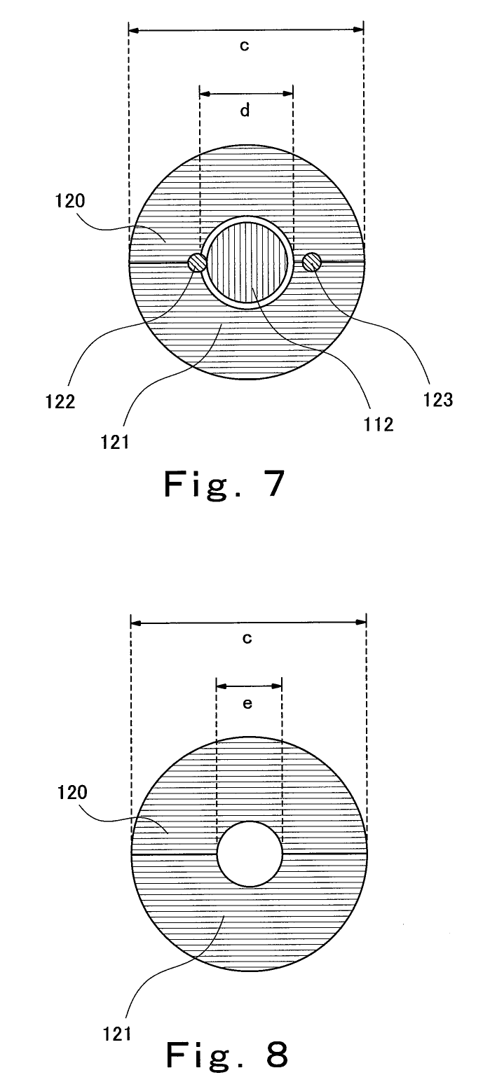

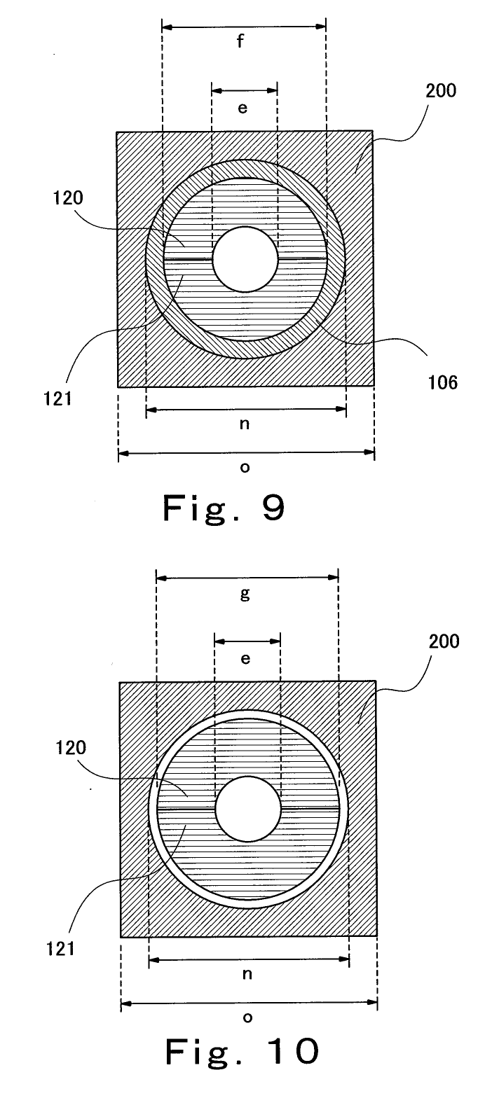

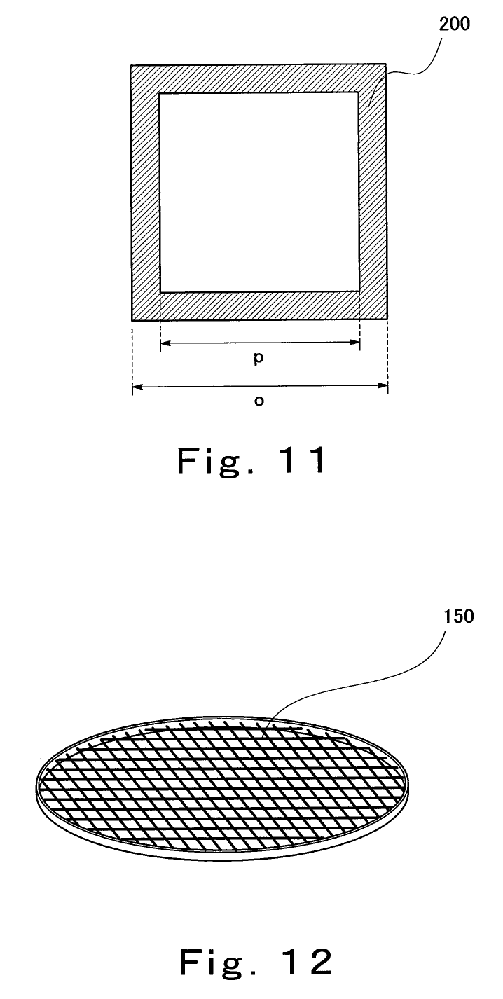

FIG. 7 is a cross-sectional view schematically showing the configuration of a cross section taken along line A-A′ of FIG. 5. FIG. 8 is a cross-sectional view schematically showing the configuration of a cross section taken along line B-B′ of FIG. 5. FIG. 9 is a cross-sectional view schematically showing the configuration of a cross section taken along line C-C′ of FIG. 5. FIG. 10 is a cross-sectional view schematically showing the configuration of a cross section taken along line D-D′ of FIG. 5. FIG. 11 is a cross-sectional view schematically showing the configuration of a cross section taken along line E-E′ of FIG. 5.

FIG. 12 is a perspective view schematically showing the configuration of an appearance of each of the first electrode and the second electrode included in the body fluid sampling device.

As shown in FIGS. 4 to 6, a semicircular cutout portion 124 is formed on a part of a joint surface of each of a first cylinder member 120 and a second cylinder member 121 such that the first electrode 112, the second electrode 113, and the separator 114 can be held by combining the first cylinder member 120 with the second cylinder member 121. Moreover, two grooves on which a first lead 122 and a second lead 123 are disposed are formed on the joint surfaces of the first cylinder member 120 and the second cylinder member 121. The depth of each groove is about 0.4 mm. Each of the first cylinder member 120 and the second cylinder member 121 can be manufactured by resin mold using a die.

A method for manufacturing the cylinder 107 and a method for arranging the first electrode 112, the second electrode 113, and the separator 114 in the suctioning passage 105a will be explained.

As shown in FIGS. 7 to 11, the cylinder 107 is formed by joining and combining the first cylinder member 120 and the second cylinder member 121. In the present embodiment, the first cylinder member 120 and the second cylinder member 121 are laterally symmetric.

First, for example, an operator sandwiches the separator 114 between the first electrode 112 and the second electrode 113 such that outer peripheries of these components are substantially flush with one another, and places these components on a table. Then, the operator places a resin plate on the first electrode 112 and the second electrode 113 sandwiching the separator 114 and applies pressure on these components by hand by utilizing the weight of the operator himself or herself, thereby combining the first electrode 112, the second electrode 113, and the separator 114. After that, the operator removes the resin plate, and places an assembly of these three components, obtained by applying the pressure, on the cutout portion 124 formed on the joint surface of the first cylinder member 120.

Next, the operator places, along lead grooves formed on the joint surfaces, the first lead 122 and the second lead 123 which electrically connect the first electrode 112 and the second electrode 113 to the voltage applying portion 115 and the detector 116. To prevent gas from leaking from the lead grooves, it is preferable that silicon sealant be lightly applied to the grooves. The first lead 122 and the second lead 123 are commercially available vinyl chloride coating copper wires, and its diameter including a coating portion is 0.8 mm. Then, the operator electrically connects the leads and the electrodes by soldering. Instead of this configuration, terminals may be separately disposed to electrically connect the electrodes and the leads.

Then, the operator combines the first cylinder member 120 on which the first electrode 112, the second electrode 113, the separator 114, the first lead 122, and the second lead 123 are placed, with the second cylinder member 121 such that the joint surfaces contact each other, and welds the second cylinder member 121 and the first cylinder member 120 by heat. Welding by ultrasound may be carried out instead of welding by heat. Thus, manufacturing the cylinder 107 and arranging the first electrode 112, the second electrode 113, and the separator 114 in the suctioning passage 105a can be carried out.

In the present embodiment, sizes a to g (see FIGS. 5 to 11) of respective portions of the first cylinder member 120 and the second cylinder member 121 are as follows. That is, in the present embodiment, a size a is 3 mm, a size b is 7 mm, a size c is 11 mm, a size d is 4 mm, a size e is 3 mm, a size f is 8 mm, and a size g is 9.5 mm.

As shown in FIG. 12, in the body fluid sampling device 100 according to the present embodiment, a mesh electrode 150 is used as each of the first electrode 112 and the second electrode 113 which are disposed to extend across the suctioning passage 105a. With this configuration, since the ratio of voids to the electrode is high, the flow of gas caused by the suctioning becomes further smooth. In addition, since the configuration of the electrode is comparatively simple, it is preferable in light of cost.

The mesh electrode 150 is configured such that a plurality of metal fine wires are woven to be orthogonal to one another. In the present embodiment, fine wires made of nickel are used as the fine wires constituting the mesh electrode 150. In the present embodiment, the thickness of each electrode is about 0.5 mm, and each electrode has a circular shape whose diameter is about 3.8 mm. Although the mesh electrode 150 having a metal ring at its outer peripheral portion is shown in FIG. 12, the mesh electrode 150 is not limited to this. For example, the mesh electrode 150 may be configured not to have the ring at its outer periphery. A commercial product can be used as the mesh electrode 150 according to the present embodiment.

In addition to the mesh electrode 150, a carbon felt, a porous carbon, or the like which is a porous electric conductor can be used as each of the first electrode 112 and the second electrode 113. In the body fluid sampling device 100 according to the present invention, the mesh electrode 150 is the most preferable electrode as each of the first electrode 112 and the second electrode 113. With this configuration, the flow of gas caused by the suctioning becomes further smooth. Moreover, with this configuration, since the configuration of the mesh electrode 150 is comparatively simple and is preferable in light of cost, the body fluid sampling device 100 can be manufactured at low cost. examples of the material constituting the first electrode 112 and the second electrode 113 are nickel, and in addition, metals, such as iron, copper, aluminum, lead, zinc, palladium, gold, platinum, alloys of these metals, and brass, and electrically-conductive resin, carbon, and the like.

Although the mesh electrode 150 formed by weaving a large number of metal fine wires is used in the present embodiment, the mesh electrode 150 is not limited to this.

FIG. 14 is a perspective view schematically showing the configuration of an appearance of a modification example of each of the first electrode and the second electrode included in the body fluid sampling device.

As shown in FIG. 14, in the present embodiment, instead of the mesh electrode 150, an electrode 800 having voids penetrating therethrough can be used as each of the first electrode 112 and the second electrode 113. The electrode 800 is obtained by mechanically forming through holes 801 on a metallic plate or carbon plate by cutting, punching, or the like. Moreover, instead of the mesh electrode 150, a mesh electrode formed by molding electrically-conductive resin in a mesh shape may be used. The first electrode 112 and the second electrode 113 do not have to have the same shape or be made of the same material, and may have different shapes and be made of different materials.

As the separator 114, general filter paper used for filtering and made of paper (cellulose) is used. In the present embodiment, the separator 114 has a thickness of about 0.1 mm, and as with the first electrode 112 and the second electrode 113, has a circular shape whose diameter is about 3.8 mm. Since the filter paper constituting the separator 114 has voids, gas easily pass through the separator 114. A commercial product can be used as the separator 114 of the present embodiment.

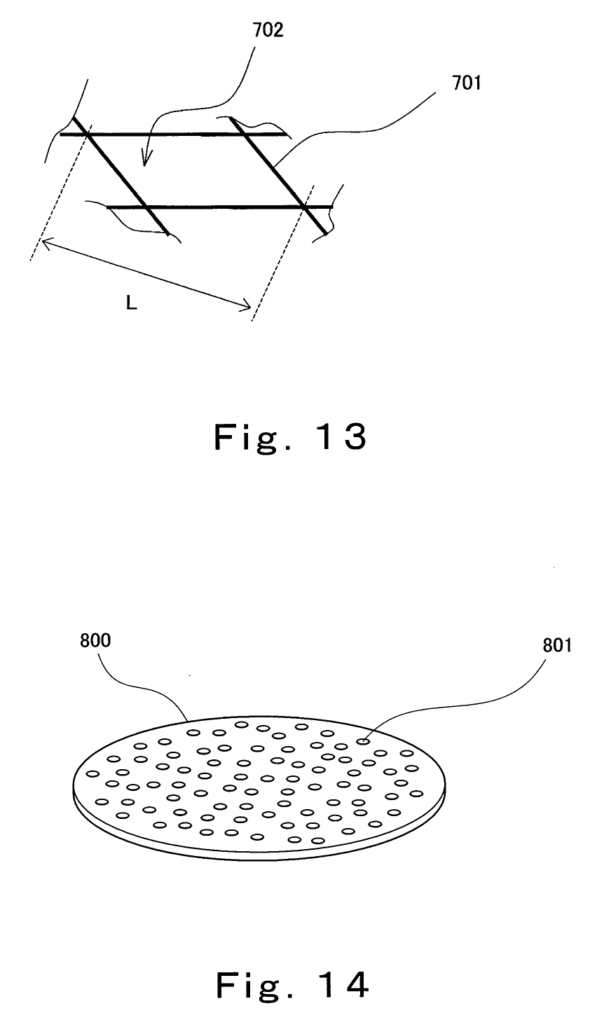

Next, the configuration of the void of each of the first electrode 112 and the second electrode 113 will be explained in reference to FIG. 13.

FIG. 13 is an enlarged perspective view schematically showing the void of each of the first electrode and the second electrode included in the body fluid sampling device.

As shown in FIG. 13, in the present embodiment, metal fine wires 701 are combined to be orthogonal to one another, thereby forming the first electrode 112 and the second electrode 113. Avoid 702 is formed at a portion surrounded by a pair of the fine wires 701 opposed to each other and a pair of the fine wires 701 orthogonal to the above pair of the fine wires 701 and opposed to each other. The width of the void 702 in the first electrode 112 and the second electrode 113 is set to about 0.4 mm at a maximum. The maximum width of the void 702 is, for example, a width indicated by a length L shown in FIG. 13.

A concept of setting the void 702 will be explained. Assuming that the separator 114 having a thickness h is impregnated with a body fluid droplet of a spherical shape whose radius is r, and the body fluid droplet becomes a body fluid droplet of a columnar shape having a height h and a diameter 2R, the diameter 2R of the body fluid droplet with which the separator 114 has been impregnated can be calculated as 2R=2(4r³/3h)^{1/2}.

Normally, the radius r of the body fluid droplet is about 0.15 mm to 3 mm, and the thickness h of the separator 114 is about 0.1 mm. Therefore, for example, in a case where the separator 114 is impregnated with the body fluid droplet having the radius r of 0.15 mm that is the lower limit, the diameter of the body fluid droplet with which the separator 114 has been impregnated is calculated to be about 0.42 mm. This is larger than the maximum value 0.4 mm of the width of the void 702 of the first electrode 112 and the second electrode 113. Therefore, the body fluid droplets with which the separator 114 has been impregnated surely contact the first electrode 112 and the second electrode 113 via the separator 114.

Therefore, in a case where the thickness h of the separator 114 is 0.1 mm, the body fluid droplets with which the separator 114 has been impregnated surely contact the first electrode 112 and the second electrode 113 via the separator 114 by setting the maximum value of the width of the void 702 to be 0.4 mm or less.

In order that the body fluid droplets with which the separator 114 has been impregnated surely contact the first electrode 112 and the second electrode 113 via the separator 114 when the maximum value of the width of the void 702 is 0.2 mm, the diameter 2R of the body fluid droplet with which the separator 114 has been impregnated may be 0.2 mm or more. Assuming that the diameter 2R of the body fluid droplet becomes 0.2 mm after the separator 114 has been impregnated with the body fluid droplet having the radius of 0.15 mm that is the lower limit, the thickness h of the separator 114 is calculated to be 0.45 mm using the above formula.

Therefore, by setting the thickness h of the separator 114 to be 0.45 mm or less when the maximum value of the width of the void 702 is set to 0.2 mm, the body fluid droplets with which the separator 114 has been impregnated surely contact the first electrode 112 and the second electrode 113 via the separator 114.

Thus, by setting the maximum value of the width of the void 702 in accordance with the thickness h of the separator 114 to be used, the first electrode 112 and the second electrode 113 can surely capture the body fluid droplets. In contrast, by setting the thickness h of the separator 114 in accordance with the maximum value of the width of the void 702 of the electrode to be used, the first electrode 112 and the second electrode 113 can surely capture the body fluid droplets.

It is preferable that the void 702 of the first electrode 112 and the second electrode 113 have the width of about 0.01 mm to 5 mm. With this configuration, since the width of the void 702 is smaller than the diameter of the droplet of the body fluid suctioned into the body fluid sampling device 100, the void 702 can effectively capture the droplet of the body fluid.

Next, the operation of the body fluid sampling device 100 according to the present embodiment will be explained in reference to FIGS. 1 to 11.

First, the operator attaches the body fluid container 200 to the body fluid container attaching portion 101. As shown in FIGS. 4 to 11, the body fluid container 200 has a rectangular solid shape and includes a space 201 which stores the body fluid. One end of the space 201 opens so as to serve as an opening 202 which is joined to the connecting portion 105 when the body fluid container 200 is attached to the body fluid container attaching portion 101. A body fluid introducing port 203 is formed on a side opposite the opening 202 side so as to be communicated with the space 201. The body fluid container 200 can be easily manufactured by injection molding of polystyrene.

Sizes h to p (see FIGS. 4 to 11) of respective portions of the body fluid container 200 used in the present embodiment are as follows. That is, in the present embodiment, a size h is 1 mm, a size i is 1 mm, a size j is 1 mm, a size k is 5.5 mm, a size l is 5.5 mm, a size m is 25 mm, a size n is 10 mm, a size o is 12 mm, and a size p is 10 mm.

Next, the operator immerses at least the body fluid introducing port 203 of the body fluid container 200 inside the body fluid obtained in a container, such as a paper cup. In this state, by pressing the body fluid suction start button 102, the controller 118 causes the suctioning portion 117 to operate. Specifically, when the operator presses the body fluid suction start button 102, the motor 110 disposed inside the body fluid sampling device 100 drives to pull up the plunger 108 inside the cylinder 107 via the plunger joint 109, thereby suctioning the gas in the space 201 of the body fluid container 200. With this, a predetermined amount (6 mL for example) of the body fluid is suctioned into the space 201 from the body fluid introducing port 203 of the body fluid container 200.

The amount of the body fluid suctioned into the body fluid container 200 is set so that the body fluid does not contact the connecting portion 105 of the body fluid sampling device 100. The suctioned body fluid is stored in the body fluid container 200 by maintaining the plunger 108 at a position at the time of completion of the suctioning of the body fluid. After the suctioning of the body fluid is completed, the body fluid introducing port 203 may be pulled up from the body fluid. At this time, if the body fluid suction start button 102 is pressed, the controller 118 causes the voltage applying portion 115 to start applying the voltage between the first electrode 112 and the second electrode 113, but the current flows little since a resistance between the first electrode 112 and the second electrode 113 is high in this state. Therefore, the current is not detected by the detector 116.

When discharging the body fluid stored in the body fluid container 200, the body fluid sampling device 100 to which the body fluid container 200 is attached, or the container, such as a paper cup, is moved such that the body fluid introducing port 203 of the body fluid container 200 is located above the container, such as a paper cup. In this state, by pressing the body fluid discharge button 103, the motor 110 drives to pull down the plunger 108 inside the cylinder 107 via the plunger joint 109. With this, the body fluid stored in the body fluid container 200 is discharged into the container, such as a paper cup. After the discharging of the body fluid is completed, the operator finally pulls out the body fluid container 200 from the body fluid sampling device 100.

Although not shown in FIGS. 1, 2, etc., the body fluid sampling device 100 according to the present embodiment includes a power supply. Used as this power supply is a battery for example. The voltage output from the battery is applied to voltage applied portions of components, such as the motor 110 to which an electric power needs to be supplied, constituting the body fluid sampling device 100. With this, the operation of the body fluid sampling device 100 is realized.

Because of carelessness or misoperation of the operator during the suctioning by the suctioning portion 117, the body fluid introducing port 203 of the body fluid container 200 may move to be located above the fluid level of the body fluid in the container, such as a paper cup. In this case, since air is suctioned together with the body fluid through the body fluid introducing port 203, the droplets of the body fluid may not be stored in the body fluid container 200 but may splash and get into the suctioning passage 105a of the body fluid sampling device 100.

In the present embodiment, the separator 114 disposed between the first electrode 112 and the second electrode 113 is impregnated with the body fluid which has been improperly suctioned into the suctioning passage 105a of the body fluid sampling device 100. In a case where the body fluid with which the separator 114 has been impregnated contacts the first electrode 112 and the second electrode 113, an electrical resistance between the first electrode 112 and the second electrode 113 lowers due to liquid junction, and thereby a current flows between the first electrode 112 and the second electrode 113. When the detector 116 detects this current, the controller 118 causes the motor 110 to stop operating, thereby stopping the suctioning by the suctioning portion 117. With this, it is possible to surely prevent the body fluid from being further suctioned and getting into the suctioning portion 117. As above, the body fluid sampling device 100 according to the present embodiment can surely detect that the body fluid has improperly got into the body fluid sampling device 100.

In the present embodiment, simultaneously with the stopping of the suctioning, the controller 118 causes the display portion 104 to display that the droplet of the body fluid has been detected. With this, the body fluid sampling device 100 informs the user that the droplet of the obtained body fluid has got into the suctioning passage 105a.

The present embodiment has explained a case where the display portion 104 visually informs that the droplet of the body fluid has got into the suctioning passage 105a. However, the present embodiment is not limited to this. The user can recognize the splashing of the body fluid by, for example, auditory information regarding the splashing of the body fluid.

As above, the body fluid sampling device 100 according to the present invention includes a notifying portion which notifies that the detector 116 has detected the electric signal. Examples of the notifying portion are a display apparatus, such as a display which displays texts, symbols, pictures, and the like, and in addition, a buzzer which emits alarm (beep sound for example) and a speaker which emits sounds. With this configuration, in a case where the detector 116 has detected the improperly suctioned body fluid, it is possible to notify the user of this improper suctioning, and therefore, the user can recognize that the misoperation has occurred during the operation of the body fluid sampling device 100. The notifying portion may display in the display portion information for urging the user to clean and/or replace members, such as the electrode, the separator and the filter, which may contact the body fluid by the improper suctioning in the body fluid sampling device 100, and the notifying portion may further inform it by sounds.

The present embodiment has explained a case where the controller 118 stops the suctioning of the suctioning portion 117. However, the present embodiment is not limited to this. For example, instead of stopping the suctioning of the suctioning portion 117 by the controller 118, the controller 118 may cause the suctioning portion 117 to stop the suctioning, and may then gently cause the motor 110 of the suctioning portion 117 to discharge the improperly suctioned body fluid. With this configuration, it is possible to minimize an intrusion of the body fluid into the suctioning portion 117 by further suctioning of the body fluid and contamination of the suctioning portion 117 by the improperly suctioned body fluid.

The present embodiment has explained a case where the suctioning portion 117 includes the stepping motor as the motor 110. However, the present embodiment is not limited to this. Instead of the stepping motor, a diaphragm pump may be used as the motor 110.

The present embodiment has explained a case where the O-ring made of rubber covered with Teflon (trademark) is used as the O-ring 106 or the like. Instead of this, the O-ring may be made of isoprene rubber, natural rubber, fluorocarbon rubber, silicone rubber, fluorocarbon resin, or the like.

### Embodiment 2

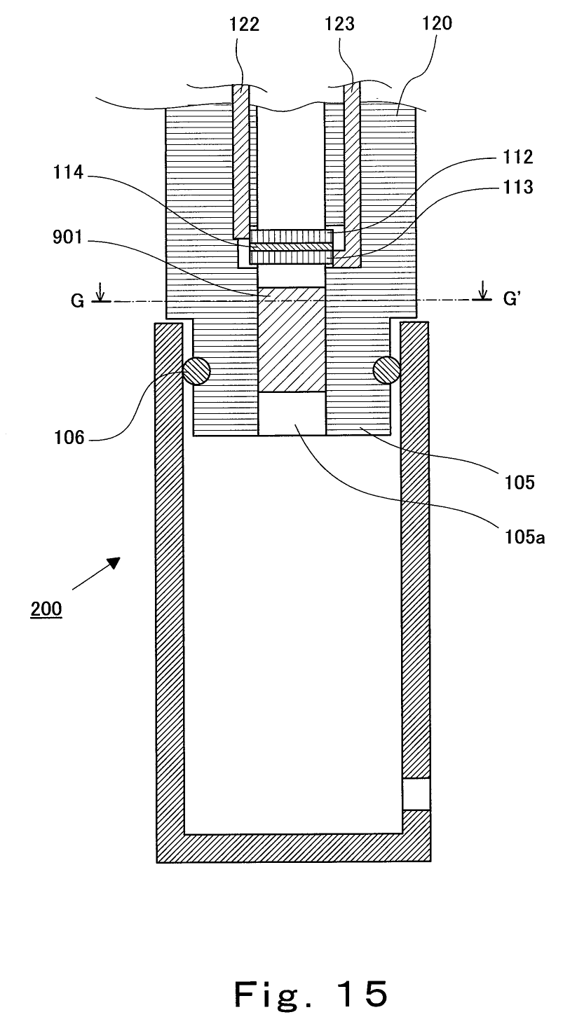

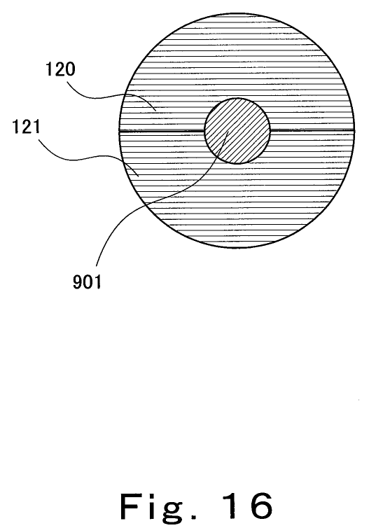

Next, Embodiment 2 of the present invention will be explained in reference to FIGS. 15 and 16.

FIGS. 15 and 16 are diagrams showing the portion including the body fluid container and the vicinity of the connecting portion of the body fluid sampling device according to Embodiment 2 when the body fluid container is connected to the connecting portion.

Specifically, FIG. 15 is a longitudinal sectional view schematically showing the portion including the body fluid container and the vicinity of the connecting portion of the body fluid sampling device according to Embodiment 2 when the body fluid container is connected to the connecting portion. FIG. 16 is a cross-sectional view schematically showing the configuration of a cross section taken along line G-G′ of FIG. 15. In FIGS. 15 and 16, the casing is omitted for convenience sake.

The body fluid sampling device according to Embodiment 2 is different from the body fluid sampling device according to Embodiment 1 in that a filter 901 is further disposed between the second electrode 113 and the tip end of the connecting portion 105 in the suctioning passage 105a. Other than this, the body fluid sampling device according to Embodiment 1 and the body fluid sampling device according to Embodiment 2 are the same in configuration as each other. Therefore, explanations of components similar to the components of the body fluid sampling device according to Embodiment 1 are omitted in the present embodiment.

The filter 901 can be easily prepared by, for example, pressure-forming commercially available nonwoven fabric of polypropylene and polyethylene in a columnar shape and cutting it. In the present embodiment, the filter 901 has a columnar shape. The filter 901 has a bottom surface whose diameter is about 3.2 mm, and has a height of about 5 mm.

In the present embodiment, the filter 901 can be disposed in the suctioning passage 105a as follows: the filter 901 is pushed into a predetermined position between the second electrode 113 and the tip end of the connecting portion 105 in the first cylinder member 120 on which the first electrode 112, the second electrode 113, the separator 114, the first lead 122 and the second lead 123 are disposed; the first cylinder member 120 and the second cylinder member 121 are combined with each other such that those joint surfaces contact each other; and the first cylinder member 120 and the second cylinder member 121 are welded by heat or the like. The filter 901 has flexibility and elasticity. Therefore, although the diameter of the filter 901 is larger than the diameter of the suctioning passage 105a, the filter 901 can be stored in the suctioning passage 105a by applying a compressive stress to the filter 901. In this case, by the stress applied to the filter 901, the filter 901 is surely fixed in the suctioning passage 105a.

In accordance with the configuration of the body fluid sampling device 100 according to the present embodiment, since the filter 901 can capture the droplets which may be generated during the suctioning of the body fluid, it is possible to reduce the possibility of the intrusion of the body fluid into the suctioning portion 117. Moreover, in a case where the droplets whose amount exceeds an amount of liquid which can be held by the filter 901 are generated, or in a case where the droplets are separated from the filter 901 due to the continuous suctioning of the body fluid, as with Embodiment 1, the body fluid droplets having been separated from the filter 901 are detected by the detector 116 using the first electrode 112 and the second electrode 113, and the suctioning by the suctioning portion 117 is stopped based on the detection result, thereby suppressing the intrusion of the droplets into the suctioning portion 117.

The present embodiment has explained a case where the second electrode 113 and the filter 901 are isolated from each other. However, the present embodiment is not limited to this.

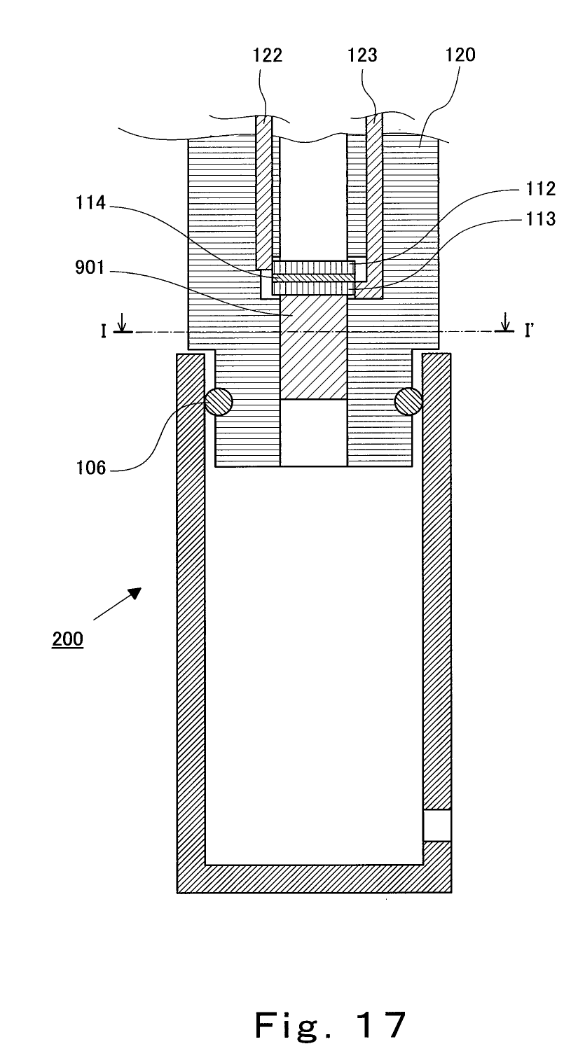

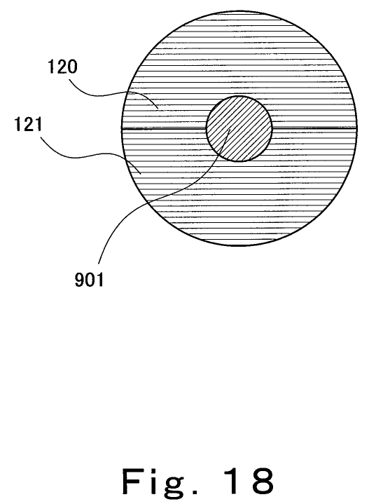

FIG. 17 relates to a modification example of the body fluid sampling device, and is a longitudinal sectional view schematically showing the portion including the body fluid container and the vicinity of the connecting portion of the body fluid sampling device when the body fluid container is connected to the connecting portion. FIG. 18 is a cross-sectional view schematically showing the configuration of a cross section taken along line I-I′ of FIG. 17.

As shown in FIGS. 17 and 18, in the body fluid sampling device 100, the filter 901 may be placed to contact the second electrode 113. With this configuration, when the filter 901 is saturated with the body fluid droplets improperly generated during the suctioning, the separator 114 is impregnated with the body fluid. Therefore, this configuration can obtain the effect of being capable of reducing the possibility of the intrusion of the body fluid into the suctioning portion 117, and the effect of being capable of detecting the improper suctioning of the body fluid into the body fluid sampling device 100 by using the first electrode 112 and the second electrode 113, as with Embodiment 1. In other respects, this configuration is the same as Embodiment 1.

### Embodiment 3

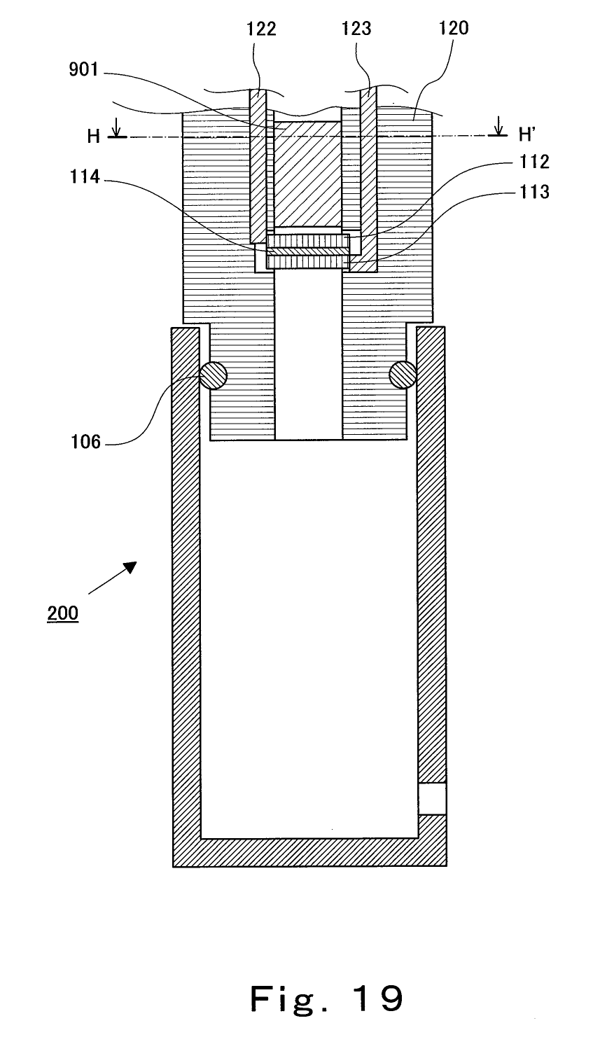

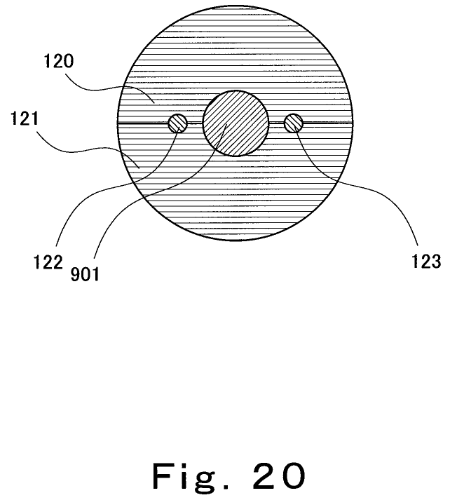

Next, Embodiment 3 of the present invention will be explained in reference to FIGS. 19 and 20.

FIGS. 19 and 20 are diagrams showing the portion including the body fluid container and the vicinity of the connecting portion of the body fluid sampling device according to Embodiment 3 when the body fluid container is connected to the connecting portion.

Specifically, FIG. 19 is a longitudinal sectional view schematically showing the portion including the body fluid container and the vicinity of the connecting portion of the body fluid sampling device according to Embodiment 3 when the body fluid container is connected to the connecting portion. FIG. 20 is a cross-sectional view schematically showing the configuration of a cross section taken along line H-H′ of FIG. 19. In FIGS. 19 and 20, the casing is omitted for convenience sake.

The body fluid sampling device according to the present embodiment is different from the body fluid sampling device 100 according to Embodiment 1 in that the filter 901 is further disposed between the first electrode 112 and the plunger 108 in the suctioning passage 105a. In other configuration, the body fluid sampling device according to the present embodiment is the same as the body fluid sampling device 100 according to Embodiment 1. Therefore, explanations of common components are omitted herein. Moreover, since the material of the filter 901, the method for creating the filter 901, the size of the filter 901, and the method for placing the filter 901 in the suctioning passage 105a are the same as those in Embodiment 2, explanations thereof are omitted.

In accordance with the configuration of the body fluid sampling device according to the present embodiment, as with Embodiment 1, since the separator 114 disposed between the first electrode 112 and the second electrode 113 is impregnated with the body fluid having been improperly suctioned into the suctioning passage 105a of the body fluid sampling device 100, it is possible to obtain the same effect as Embodiment 1. Further, in accordance with the present embodiment, even in a case where a large amount of the body fluid whose amount exceeds an amount of liquid which can be held by the separator 114 is suctioned in a period from when the body fluid is suctioned into the suctioning passage 105a until when the suctioning of the suctioning portion 117 stops, the body fluid is captured by the filter 901 disposed between the first electrode 112 and the plunger 108, thereby suppressing the intrusion of the body fluid into the suctioning portion 117.

In the present embodiment, the first electrode 112 and the filter 901 are isolated from each other. However, the present embodiment is not limited to this. To be specific, as shown in FIG. 17, in the present embodiment, in the body fluid sampling device 100, the filter 901 may be placed to contact the first electrode 112. Moreover, the present embodiment may be combined with Embodiment 2. To be specific, the filters 901 may be disposed both above the first electrode 112 and under the second electrode 113. In other respects, the present embodiment is the same as Embodiments 1 and 2.

### Embodiment 4

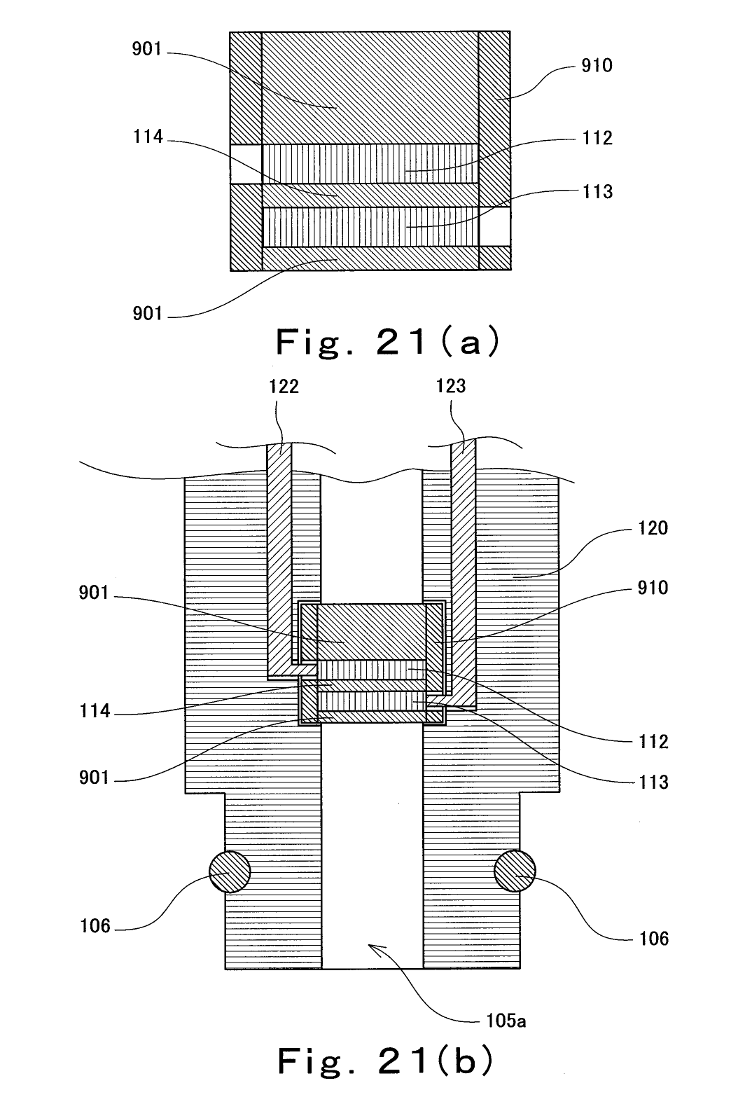

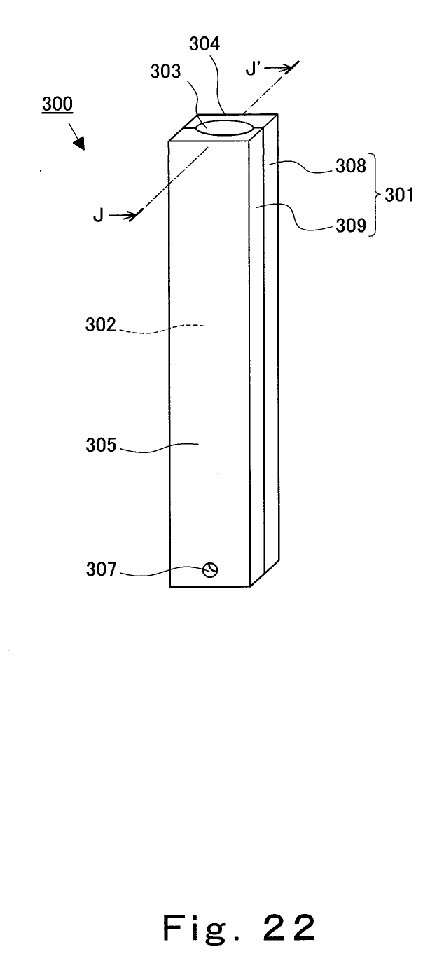

Next, Embodiment 4 of the present invention will be explained in reference to FIG. 21.

FIGS. 21 are longitudinal sectional views schematically showing the configuration of major components of the body fluid sampling device according to Embodiment 4. FIG. 21(a) is a longitudinal sectional view schematically showing the arrangement of the first electrode, the second electrode, the separator and the filter. FIG. 21(b) is a longitudinal sectional view schematically showing the arrangement of an assembly of the first electrode, the second electrode, the separator and the filter.

As shown in FIG. 21, the body fluid sampling device according to the present embodiment is different from the body fluid sampling devices according to Embodiments 2 and 3 in that the filter 901, the first electrode 112, the second electrode 113 and the separator 114 are integral to form one member. In other respects, the present embodiment is the same as Embodiments 2 and 3, and a detailed explanation thereof is omitted.

In the present embodiment, the member formed of the filter 901, the first electrode 112, the second electrode 113 and the separator 114 can be manufactured by wrapping the first electrode 112, the second electrode 113 and the separator 114 in a commercially available nonwoven fabric made of polypropylene and polyethylene that are materials of the filter 901, pressure-forming this wrapped assembly in a columnar shape, and cutting this columnar assembly. In this case, the assembly of the first electrode 112, the second electrode 113, the separator 114 and the filter 901 does not have to have a columnar shape, but may have a spherical shape. Instead of this manufacturing method, the assembly of the first electrode 112, the second electrode 113, the separator 114 and the filter 901 can also be manufactured in such a manner that: resin, i.e., the above-described material of the filter 901 is formed like a sponge, cutout portions are formed on the sponge, and the first electrode 112, the second electrode 113 and the separator 114 are inserted into the cutout portions. Alternatively, the assembly of the first electrode 112, the second electrode 113, the separator 114 and the filter 901 may be manufactured using the method described in Embodiment 1 in such a manner that: the first electrode 112, the second electrode 113, and the separator 114 are pressure-formed to have a columnar shape, the column is cut, and the obtained component is sandwiched between two columnar filters 901.

In the present embodiment, outer surfaces of the filter 901, the first electrode 112, the second electrode 113 and the separator 114 are surrounded by a surrounding member 910. Thus, using the surrounding member 910, it is possible to provide the member formed by integrating the filter 901, the first electrode 112, the second electrode 113 and the separator 114. Examples of the material constituting the surrounding member 910 are resins, such as ABS resin, polypropylene, fluorocarbon resin (Teflon (trademark) for example), isoprene rubber, natural rubber, silicone rubber, and fluorocarbon rubber.

As shown in FIG. 21(b), the assembly of the first electrode, the second electrode, the separator and the filter is disposed between the first cylinder member 120 and the second cylinder member 121 so as to extend across the suctioning passage 105a. Moreover, the first electrode 112 and the first lead 122 are electrically connected to each other, and the second electrode 113 and the second lead 123 are electrically connected to each other.

As above, the present embodiment describes the body fluid sampling device 100 of a cartridge type in which the first electrode, the second electrode, the separator and the filter are integrally replaceable (detachable) with respect to the body fluid sampling device 100. With this configuration, in a case where it is necessary to replace, for example, the filter 901 and two electrodes, this replacing can be easily carried out. With this, it is possible to provide the cartridge type body fluid sampling device 100 whose convenience has been improved. In the case of replacing, for example, the filter 901 and two electrodes, the first cylinder member 120 and the second cylinder member 121 may be exposed, and the first cylinder member 120 and the second cylinder member 121 may be then separated from each other. To be specific, in the present embodiment, since the assembly of the first electrode, the second electrode, the separator and the filter is configured to be detachable from the inside of the suctioning passage 105a, it is possible to easily replace the assembly when it is necessary to replace, for example, the filter 901 and a pair of electrodes.

In accordance with the configuration of the body fluid sampling device 100 according to the present embodiment, since the filter 901(cartridge) including therein the first electrode 112, the second electrode 113 and the separator 114 can be handled as a single integral component, and this further improves the productivity of the body fluid sampling device, the configuration of the body fluid sampling device 100 according to the present embodiment is very preferable. The present embodiment has explained the cartridge type body fluid sampling device 100 in which the first electrode 112, the second electrode 113, the separator 114 and the filter 901 are integral with each other. However, if at least the first electrode 112, the second electrode 113 and the separator 114 are integrated in the cartridge, these components except for the filter 901 can be handled as a single component. In this case, as with Embodiment 1, it is possible to obtain the configuration in which the filter is not used. Also, in a case where the filter 901 does not contact the electrodes and in a case where the filter 901 contacts only one of the electrodes as described in Embodiments 2 to 3, it is possible to use the cartridge in which the first electrode 112, the second electrode 113 and the separator 114 are integral with each other.

In accordance with the configuration of the body fluid sampling device 100 according to the present embodiment, the body fluid with which the filter 901 has been impregnated is not discharged to an outside of the filter 901, but can contact both electrodes. Therefore, in accordance with the present embodiment, in addition to the effect of capturing the droplets by the filter 901, it is possible to further surely detect the generation of the droplets even if the amount of the body fluid droplets is much smaller than Embodiments 2 and 3.

Embodiments 2 to 4 have explained one example in which the nonwoven fabric made of polypropylene and polyethylene is used as the filter 901. Instead of this, it is possible to use, as the filter 901, fabrics, such as vegetable fiber of cotton or the like, animal hair, nylon, acryl and polyester, formed in a spherical shape, columnar shape or nonwoven fabric form, cloth of the above fabrics, or resin of the above synthetic fiber formed like a sponge.

Regarding the arrangement of the cartridge, instead of the arrangement described in Embodiments 2 to 4, a cutout portion having substantially the same shape as the cartridge may be formed on a part of the joint surfaces of the first cylinder member 120 and the second cylinder member 121, and the cartridge may be disposed in this cutout portion. Moreover, regarding the arrangement of the cartridge, the first cylinder member 120 and the second cylinder member 121 may be combined with each other such that their joint surfaces contact each other, the first cylinder member 120 and the second cylinder member 121 may be welded by heat or the like, and the cartridge may be pushed into the formed suctioning passage 105a. Even with this configuration, it is possible to obtain the same effects as in the present embodiment. Other than this, the present embodiment is the same as Embodiments 1 to 3.

### Embodiment 5

Next, Embodiment 5 of the present invention will be explained in reference to the drawings.

The present embodiment and Embodiment 6 described below will explain a body fluid measuring device which uses the body fluid sampling device 100 explained in Embodiments 1 to 4. Referring to FIGS. 22 to 25, the present embodiment will explain a case where the body fluid as a sample is urine, and the specific substance to be measured is human albumin. Examples of the body fluid in the present specification are serum, blood plasma, blood, urine, interstitial fluid, and lymph fluid. Example of the specific substance are albumin, hCG, LH, CRP, and IgG.

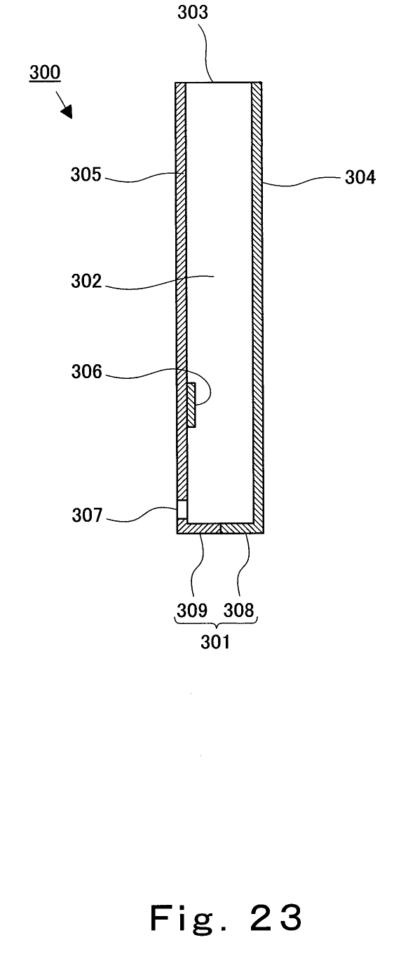

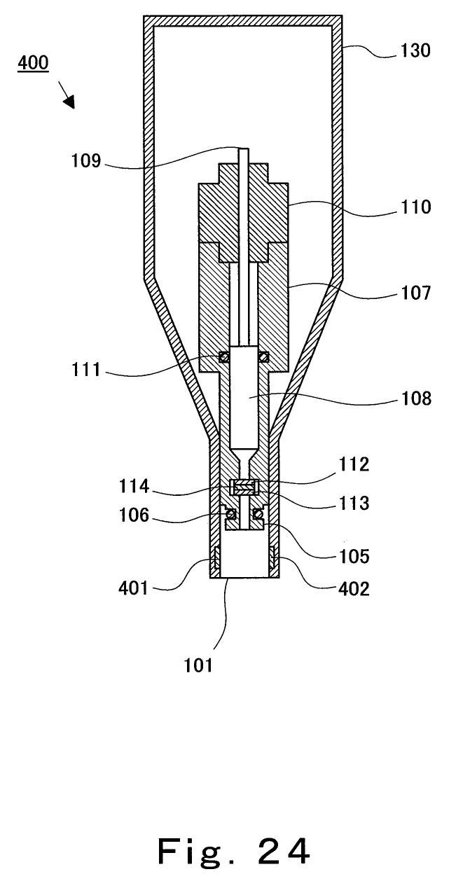

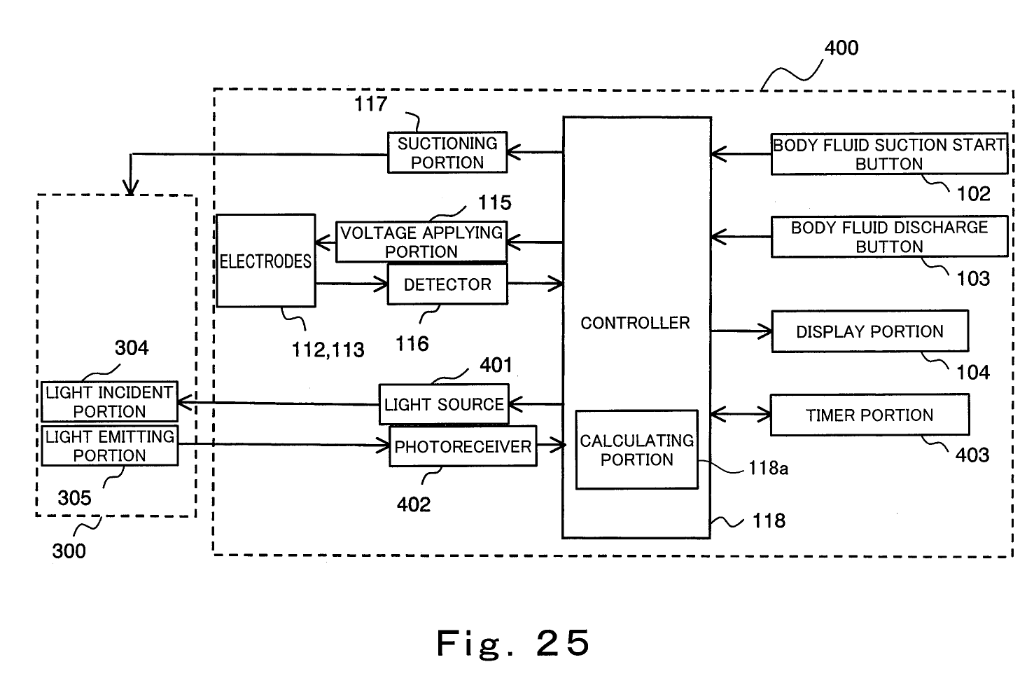

FIG. 22 is a perspective view schematically showing the configuration of an appearance of the body fluid container used in Embodiment 5. FIG. 23 is a cross-sectional view schematically showing the configuration of a cross section taken along line J-J′ of FIG. 22. FIG. 24 is a cross-sectional view schematically showing the configuration of a cross section of the body fluid sampling device according to Embodiment 5. FIG. 25 is a block diagram schematically showing an internal configuration of the body fluid measuring device.

First, the configuration of the body fluid container used in the present embodiment will be explained in reference to FIGS. 22 and 23.

As shown in FIGS. 22 and 23, a body fluid container 300 used in the present embodiment includes a base body 301 made of transparent polystyrene and having a rectangular solid shape. The base body 301 includes a space 302 which stores the body fluid. One end of the space 302 opens so as to serve as an opening 303 which is joined to the connecting portion when the body fluid container 300 is attached to the body fluid container attaching portion. Moreover, among four surfaces constituting outer surfaces of the base body 301, one surface functions as a light incident portion 304, and a surface opposed to the above surface functions as a light emitting portion 305.

As shown in FIG. 23, a reagent holding portion 306 holding a reagent for optical measurement is disposed on an inner wall surface, which surrounds the space 302 and corresponds to an inner side of a surface on which the light emitting portion 305 is disposed. Moreover, a body fluid introducing port 307 through which the sample, i.e., the body fluid is introduced into the space 302 is formed on the surface on which the light emitting portion 305 is disposed. In the present embodiment, the base body 301 is 12 mm long, 12 mm wide, and 25 mm high, as with the body fluid container according to Embodiment 1.

As above, the body fluid container 300 includes: the reagent holding portion 306 which holds the reagent necessary to carry out the optical measurement; the light incident portion 304 on which incident light is incident; and the light emitting portion 305 which emits emitting light from an inside of the body fluid container toward an outside of the body fluid container.

Moreover, it is preferable that the light incident portion 304 and the light emitting portion 305 be made of an optically transparent material or a material which does not substantially absorb visible light. Examples of the material constituting the light incident portion 304 and the light emitting portion 305 are polystyrene, and in addition, quartz, glass, and polymethylmethacrylate. In a case where the body fluid container 300 is formed to be disposable, it is preferable that the light incident portion 304 and the light emitting portion 305 be made of polystyrene in order to suppress cost.

Next, a method for manufacturing the body fluid container will be explained in reference to FIGS. 22 and 23.

Each of a first member 308 and a second member 309 is made of transparent polystyrene, and has a concave portion. By combining the first member 308 and the second member 309 with each other, their concave portions are integrated to form the space 302, thereby constituting the base body 301. Each of the first member 308 and the second member 309 can be obtained by molding using a die. In this case, a known resin molding technique may be used.

First, the reagent holding portion 306 is formed on the bottom surface of the concave portion of the second member 309. More specifically, in the present embodiment, a certain amount of aqueous solution containing an antibody to human albumin that is the reagent used for the optical measurement is dropped using a microsyringe or the like, thereby being applied to a predetermined position on the bottom surface of the concave portion of the second member 309. This is placed at about a room temperature to 30° C. to evaporate moisture. Thus, the dried reagent is formed as the reagent holding portion 306 at a predetermined position on the bottom surface of the concave portion of the second member 309. The reagent holding portion 306 does not have to be formed on the bottom surface of the concave portion, and may be formed on a bottom (in the vicinity of the body fluid introducing port 307) of the space 302 of the body fluid container 300. For example, the concentration of the aqueous solution containing the antibody is 1.1 mg/dL, the amount of the aqueous solution dropped is 0.05 mL, and the area where the aqueous solution is dropped is 1 cm².

The concentration and amount of the aqueous solution containing the reagent to be applied are appropriately determined depending on a necessary device characteristic and a spacial limitation of a position where the second member 309 is formed. Moreover, the position and area of the reagent holding portion 306 on the second member 309 are suitably and appropriately determined in view of, for example, the solubility of the reagent with respect to the sample and the positions of the light incident portion and the light emitting portion. The antibody to human albumin can be obtained by a conventionally known method. For example, antiserum of a rabbit immunized against human albumin is refined by protein A column chromatography, and dialysis is carried out using a dialysis tube, thereby obtaining an anti-human albumin antibody.

As above, in the case of using a body fluid measuring device 400, it is preferable that the reagent contained in the reagent holding portion 306 of the body fluid container 300 contain an antibody or an enzyme. Moreover, it is preferable that the reagent be contained in a dry state in the reagent holding portion 306 of the body fluid container 300 and be placed so as to dissolve in the sample when the sample is supplied to the body fluid container 300. For example, a porous carrier formed from glass fiber, filter paper or the like is impregnated with the solution containing the reagent, and then is dried, thereby causing the carrier to hold the reagent. Then, the porous carrier holding the reagent is disposed in the body fluid container 300. As described above, the method for placing the reagent in the reagent holding portion 306 by directly applying the solution of the reagent on the wall surface on which the reagent holding portion 306 is to be formed, and drying the solution of the reagent, is the most preferable method in light of cost reduction.

Since the antibody as the reagent can be prepared by a known method, it is advantageous in that the reagent can be prepared easily. For example, a mouse, a rabbit or the like is immunized using, as an antigen, protein, such as albumin, or hormone, such as hCG or LH, thereby obtaining the antibody to the antigen. Examples of this antibody are an antibody to protein, such as albumin, contained in urine and an antibody to hormone, such as hCG or LH, contained in urine. According to need, a compound, such as polyethylene glycol, which accelerates agglutination of the antigen and the antibody may be caused to coexist with the antibody in the vicinity of the antibody in the body fluid container.

Since the enzyme as the reagent highly selectively catalyzes the reaction of a specific compound, it is possible to realize a highly selective measurement with respect to the specific compound in the sample. As the enzyme, in light of selectivity and reactivity, a conventionally known optimal enzyme is selected depending on the compound to be measured. The enzyme can be obtained from commercial products. Examples of the enzyme are glucose oxidase, glucose dehydrogenase, alcohol oxidase, cholesterol oxidase, the other oxidation-reduction enzymes, and the like. In this case, the optical measurement can be carried out stably by causing a pigment or a pigment source, which colors or fades as a result of an enzyme reaction, to coexist with the enzyme. By using cholesterol esterase combined with cholesterol oxidase, it is possible to detect cholesterol ester. These enzymes can be obtained from commercial products.

The body fluid container 300 is assembled by joining the first member 308 and the second member 309 obtained as above. In this case, adhesive, such as epoxy resin, is applied to connecting portions of respective members, and the members are bonded to one another and left as they are. By adequately drying the connecting portions, the body fluid container 300 is assembled. Instead of this assembling method, respective members may be adhered to each other, and the connecting portions may be welded by heat or ultrasound using a commercially available welding machine. Thus, the body fluid container 300 can be obtained.

Next, the configuration of the body fluid measuring device 400 according to the present embodiment will be explained in reference to FIGS. 24 and 25.

The body fluid measuring device 400 according to the present embodiment is different from the body fluid sampling device 100 according to Embodiment 1 in that included in the body fluid container attaching portion 101 are: a light source 401 which emits incident light incident on the light incident portion 304 of the body fluid container 300 attached to the body fluid container attaching portion 101; and a photoreceiver 402 which receives emitting light having been emitted from the light emitting portion 305, and in that a timer as a timer portion 403 which measures time is included. In other respects, the configurations of the body fluid measuring device 400 and the body fluid sampling device 100 according to Embodiment 1 are the same as each other. Therefore, in the following explanation, same reference numbers are used for same components, and explanations thereof are omitted.

In the present embodiment, used as the light source 401 is a semiconductor laser which emits light having a wavelength of 650 nm. Instead of the semiconductor laser, a light emitting element, such as a light emitting diode (LED), may be used as the light source 401. In the present embodiment, the measurement by immuno-nephelometry is applied, and the light having the wavelength of 650 nm is irradiated and received. However, this wavelength can be suitably and appropriately determined depending on a measurement method and a measurement target.

In the present embodiment, used as the photoreceiver 402 is a photodiode. Instead of the photodiode, a light receiving element, such as a charge coupled device (CCD) or a photomultimeter, may be used as the photoreceiver 402.

As shown in FIG. 24, in the body fluid measuring device 400, the light source 401 and the photoreceiver 402 are disposed in the vicinity of the body fluid container attaching portion 101 of the casing 130 so as to be coaxially opposed to each other (such that the photoreceiver 402 can efficiently receive the light having been emitted from the light source 401).

In the present embodiment, the light incident portion 304 and the light emitting portion 305 used for the optical measurement among four surfaces constituting the outer surfaces of the base body 301 do not have to be opposed to each other. That is, appropriate surfaces can be suitably used depending on the type of the optical measurement. For example, in the case of measuring scattering of light, the light incident portion 304 and the light emitting portion 305 may be orthogonal to each other. In this case, the light source 401 and the photoreceiver 402 may be disposed at appropriate positions corresponding to the light incident portion 304 and the light emitting portion 305 which are orthogonal to each other.

Although not shown in FIG. 25, the controller 118 of the body fluid measuring device 400 according to the present embodiment includes a memory. The memory prestores a calibration curve indicating a relationship between the concentration of human albumin that is the specific substance of the measurement target and the intensity of the emitting light received by the photoreceiver 402. In the present embodiment, the calculating portion 118a included in the controller 118 of the body fluid measuring device 400 is appropriately configured to also function as a calculating unit which measures the amount of the specific substance in the body fluid based on the light having been received by the photoreceiver 402.

Next, the operation of the body fluid measuring device 400 according to the present embodiment will be explained. Since the method for attaching the body fluid container 300 to the body fluid measuring device 400, the method for suctioning the body fluid into the body fluid container 300, the method for detecting improper suctioning of the body fluid into the suctioning passage 105a, and the method for discharging the body fluid from the body fluid container 300 are the same as those of Embodiment 1, explanations thereof are omitted.

In a case where the operator introduces urine, i.e., a body fluid sample into the space 302 of the body fluid container 300, the introduced urine dissolves the anti-human albumin antibody, i.e., the dry-state reagent held by the reagent holding portion 306. Here, an immune reaction between the anti-human albumin antibody and human albumin, i.e., the antigen in the urine proceeds. Meanwhile, when the introduction of the urine into the space 302 of the body fluid container 300 is completed, the controller 118 causes the timer as the timer portion 403 to start timing.

Next, when the controller 118 determines based on a signal supplied from the timer portion 403 that a predetermined time (two minutes for example) has passed since the completion of the introduction of the urine into the space 302 of the body fluid container 300, the controller 118 causes the light source 401 to execute light irradiation.

After the light is emitted from the light source 401, the light passes through the light incident portion 304 of the body fluid container 300, is incident in the space 302, is transmitted through and scatters in the urine, and is emitted from the light emitting portion 305. The photoreceiver 402 receives this light for a predetermined time (three minutes for example).

Then, the controller 118 reads out and refers to the calibration curve prestored in the memory, and thereby converts the intensity of the emitting light having been received by the photoreceiver 402 into the concentration of human albumin. This conversion is carried out by the calculating portion 118a included in the controller 118. The controller 118 causes the display portion 104 to display the concentration of human albumin obtained by the conversion carried out by the calculating portion 118a. Since the display portion 104 displays the concentration of human albumin, the user recognizes the completion of the measurement of the concentration of human albumin. Thus, it is possible to carry out the optical measurement of the body fluid using the body fluid measuring device 400 and the body fluid container 300 according to the present embodiment.

### Embodiment 6

Next, Embodiment 6 of the present invention will be explained in reference to the drawings.

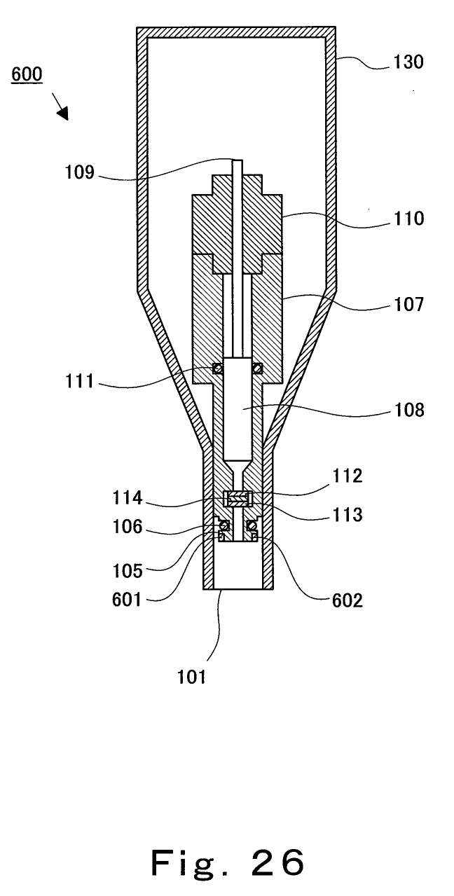

Referring to FIGS. 26 to 30, the present embodiment will explain a case where the body fluid as the sample is urine, and the specific substance as the measurement target is glucose.

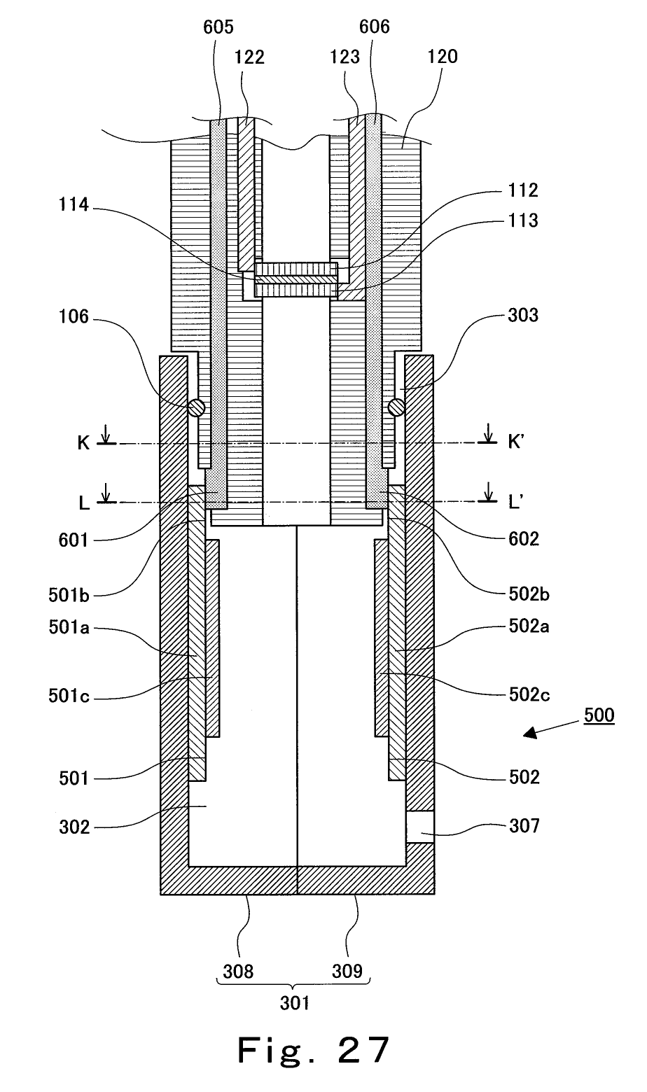

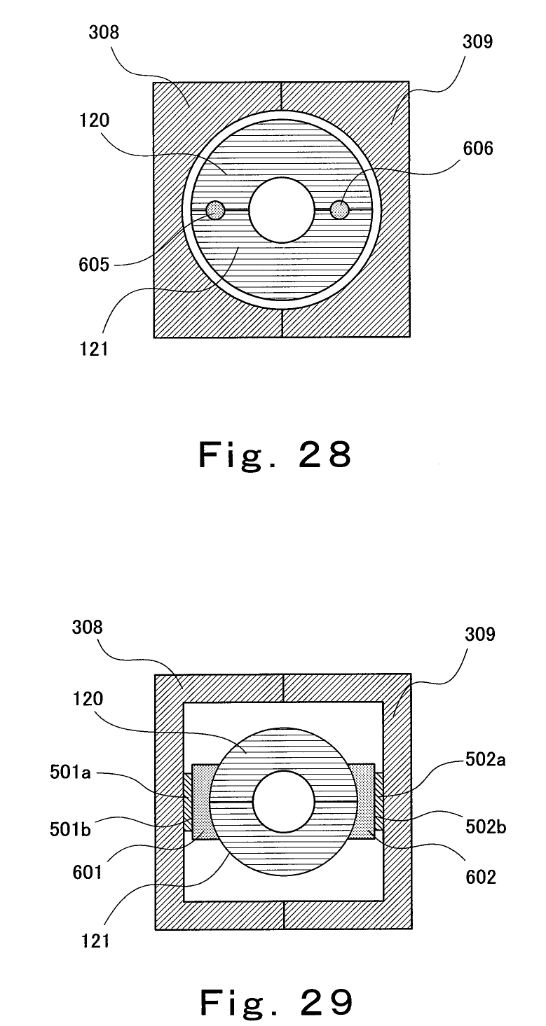

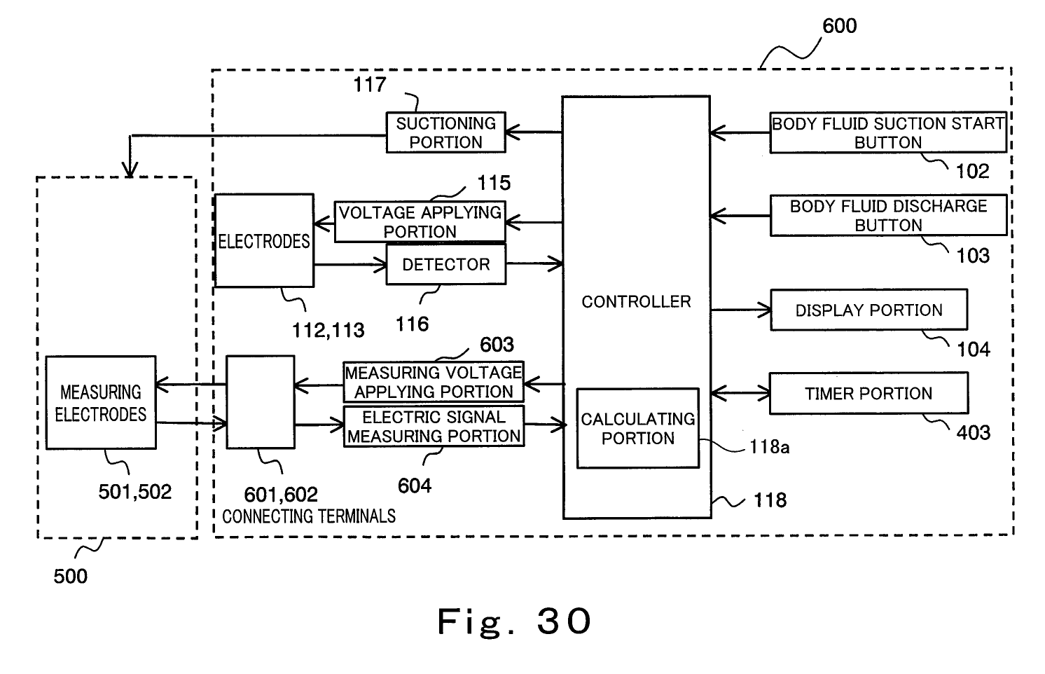

FIG. 26 is a cross-sectional view schematically showing the configuration of a cross section of the body fluid measuring device according to Embodiment 6. FIG. 27 is a longitudinal sectional view schematically showing the portion including the body fluid container and the vicinity of the connecting portion of the body fluid measuring device when the body fluid container is connected to the connecting portion. FIG. 28 is a cross-sectional view schematically showing the configuration of a cross section taken along line K-K′ of FIG. 27. FIG. 29 is a cross-sectional view schematically showing the configuration of a cross section taken along line L-L′ of FIG. 28. FIG. 30 is a block diagram schematically showing an internal configuration of the body fluid measuring device. In FIGS. 27 to 29, the casing 130 is omitted for convenience sake.

First, the configuration of the body fluid container used in the present embodiment will be explained in reference to FIGS. 27 to 29.

A body fluid container 500 used in the present embodiment includes the base body 301 made of transparent polystyrene and having a rectangular solid shape. The base body 301 includes the space 302 which stores the body fluid. One end of the space 302 opens so as to serve as the opening 303 which is joined to the connecting portion when the body fluid container 500 is attached to the body fluid container attaching portion. A first measuring electrode 501 is disposed on one of four surfaces surrounding the space 302, and a second measuring electrode 502 is disposed on a surface opposed to the above surface on which the first measuring electrode 501 is disposed. A body fluid introducing port 307 through which the sample, i.e., the body fluid is introduced into the space 302 is formed at a lower portion of the surface on which the second measuring electrode 502 is formed. In the present embodiment, the base body 301 is 12 mm long, 12 mm wide, and 25 mm high, as with the body fluid container according to Embodiment 1.

Next, a method for manufacturing the body fluid container 500 will be explained.

Each of the first member 308 and the second member 309 is made of transparent polystyrene and has the concave portion. The first member 308 and the second member 309 are combined with each other such that their concave portions are opposed to each other, thereby constituting the base body 301. Each of the first member 308 and the second member 309 can be easily obtained by molding using a die. Applied as this molding may be a known resin molding technique.

First, a first electrically-conductive portion 501a is disposed on the concave portion of the first member 308. In this case, an acryl resin mask having a void which is the same in shape as the first electrically-conductive portion 501a is disposed at an upper portion of the first member 308, gold is sputtered via the mask, and the mask is removed, thereby forming the first electrically-conductive portion 501a. Instead of the sputtering, the first electrically-conductive portion 501a can also be formed by deposition in accordance with the same procedure as above.

Next, a second electrically-conductive portion 502a is manufactured at the concave portion of the second member 309 by the same manufacturing method as the first electrically-conductive portion 501a. As with the first electrically-conductive portion 501a, the second electrically-conductive portion 502a may be manufactured by sputtering or deposition. Thus, a pair of the first electrically-conductive portion 501a and the second electrically-conductive portion 502a are formed on the inner wall surface of the body fluid container 500 so as to be opposed to each other.

The size of each of the first electrically-conductive portion 501a and the second electrically-conductive portion 502a is not especially limited. For example, each of the first electrically-conductive portion 501a and the second electrically-conductive portion 502a may have a width of about 2 mm, a length of about 25 mm, and a thickness of about 5 um. In the present embodiment, to define the size (the length, or the area of an exposing portion of the electrically-conductive portion) of each of the measuring electrodes 501 and 502 and leads 501b and 502b, covers 501c and 502c made of insulating resin are attached to cover portions other than the measuring electrodes 501 and 502 and the leads 501b and 502b. Used as each of the covers 501c and 502c may be, for example, a PET membrane which has a width of about 10 mm, a length of about 15 mm and a thickness of about 0.1 mm, and to which acrylic adhesive is applied. Moreover, the cover 501c and the cover 502c are disposed such that the length of each of the measuring electrodes 501 and 502 and the leads 501b and 502b is, for example, 5 mm. The material type, area, thickness, shape, position, and the like of each of the first electrically-conductive portion 501a, the second electrically-conductive portion 502a, the measuring electrodes 501 and 502, and the leads 501b and 502b may be suitably and appropriately adjusted depending on the characteristics of necessary devices, the position of an optical measurement system, and the like.

Next, glucose oxidase that is the enzyme and osmium complex that is an electron carrier are fixedly held by the surface of the first measuring electrode 501 using a known means. Specifically, a solution of polyvinylimidazole to which dibipyridinium osmium chloride is coordinated is mixed with a solution of glucose oxidase, this mixture is applied to the upper portion of the first measuring electrode 501, polyethylene glycol diglycidyl ether that is an amine crosslinking agent is added thereto, and these are mixed with each other. After the mixture is left as it is for about an hour, the surface of the electrode is cleaned using distilled water.

The first member 308 and the second member 309 obtained as above are joined to each other to assemble the body fluid container 500. In this case, adhesive, such as epoxy resin, is applied to the joint surfaces of the first member 308 and the second member 309. After that, the first member 308 and the second member 309 are bonded to each other and lest as they are to adequately dry the connecting portion, thereby assembling the body fluid container 500. Instead of using the adhesive, such as epoxy resin, respective members may be caused to contact one another, and a commercially available welding machine may be used to weld contacting portions of the respective members by heat or ultrasound. Thus, the body fluid container 500 can be obtained.

It is preferable that the material of each of the first electrically-conductive portion 501a and the second electrically-conductive portion 502a be a material containing at least one of gold, platinum, palladium, an alloy of gold, platinum and palladium, a mixture of gold, platinum and palladium, and carbon. Since these materials are chemically and electrochemically stable, stable measurement can be realized.

It is preferable that each of the measuring electrodes 501 and 502 be suitable for the measurement of the concentration of the specific compound or ion contained in the body fluid. With this configuration, it is possible to accurately measure the concentration of the specific compound or ion contained in the body fluid. For example, by using a glass electrode or the like as the measuring electrode, it is possible to accurately measure the concentration of sodium ion contained in the body fluid.

It is preferable that each of the measuring electrodes 501 and 502 include a membrane sensitive to a specific ion contained in the body fluid. With this configuration, it is possible to accurately measure the concentration of the specific ion contained in the body fluid. As an ion sensitive membrane, it is possible to use an ion sensitive membrane which selectively allows any one of sodium ion, potassium ion, lithium ion, magnesium ion, calcium ion, chloride ion, ammonium ion, hydrogen ion and the like, to be transmitted therethrough.

As a compound constituting the ion sensitive membrane, it is possible to use a known compound selected depending on the type of the ion which is transmitted through the ion sensitive membrane. For example, as the compound constituting the ion sensitive membrane, it is possible to use a clathrate compound having ion selectivity explained below.

In the case of sodium ion, one example of the compound is Bis[(12-crown-4)methyl]2,2-dibenzomalonate.

In the case of potassium ion, one example of the compound is Bis[(benzol5-crown-5)4-methyl]pimelate.

In the case of lithium ion, one example of the compound is phosphododecyl-14-crown-4.

In the case of magnesium ion, one example of the compound is 4,13-bis[N-(1-adamantyl)carbamoylacetyl]-8-tetradecyl-1,7,10,16-tetraoxa-4,13-diazacy clooctadecane.

In the case of calcium ion, one example of the compound is 4,16-bis(N-octadecylcarbamoyl)-3-octbutyryl-1,7,10,13,19-pentaoxa-4,16-diazacyclohen icosane.

In the case of chloride ion, one example of the compound is 2,7-Di-tert-butyl-9,9-dimethyl-4,5-bis(N-n-butylthioureylene)xanthene.

In the case of ammonium ion, one example of the compound is 2,6,13,16,23,26-hexaoxaheptacyclo[25.4.4.4^{7,12}.4^{17,22}.0^{1,17}.0^{7,12}.0^{17,22}]tritetracontane.

These compounds can be obtained as commercial products from, for example, Dojindo Laboratories.

A method for forming the ion sensitive membrane on the upper portion of each of the measuring electrodes 501 and 502 is, for example, as follows: the clathrate compound, plasticizer, an anion remover, and a high polymer compound, such as PVC, are dissolved in an organic solvent; the obtained mixture solution is applied to the upper portion of each measuring electrode; and the applied mixture solution is air-dried.

Moreover, each of the measuring electrodes 501 and 502 may be a field effect transistor (FET) formed by using elements, such as silicon. Moreover, it is preferable that a reference electrode, such as Ag/AgCl or a saturated calomel electrode, whose potential is stable be used as one of the measuring electrodes or as a third electrode.

As described above, it is preferable that the enzyme be held by the surface of the first measuring electrode 501. With this configuration, since the enzyme highly selectively catalyzes the reaction of the specific compound, it is possible to realize a highly selective measurement with respect to the specific compound in the body fluid. As the enzyme, in light of selectivity and reactivity, a conventionally known optimal enzyme is selected depending on the compound to be measured. The enzyme can be obtained from commercial products.

Examples of the enzyme are glucose oxidase, and in addition, glucose dehydrogenase, alcohol oxidase, cholesterol oxidase, the other oxidation-reduction enzymes, and the like. By using these enzymes, it is possible to preferably measure glucose, alcohol, cholesterol, or the like as the specific compound in the body fluid. Moreover, by using cholesterol esterase combined with cholesterol oxidase, it is possible to detect cholesterol ester. These enzymes can be obtained from commercial products. In the present embodiment, as described above, it is preferable that the enzyme do not dissolve in the body fluid and be fixed to the electrode. With this configuration, even if the amounts of the body fluid samples vary, it is possible to carry out a highly precise measurement.

As described above, in the present embodiment, the electron carrier which realizes transportation of electrons between the enzyme and the measuring electrode is used according to need. Examples of the electron conductor are osmium complex, and in addition, ferricyanide/ferrocyanide ion, ferrocene derivative, ruthenium complex, quinone derivative, phenazine derivative, and phenothiazine derivative.

Next, the configuration of a body fluid measuring device 600 according to the present embodiment will be explained in reference to FIGS. 26 to 30.

The body fluid measuring device 600 according to the present embodiment is different from the body fluid sampling device 100 according to Embodiment 1 in that: a first connecting terminal 601 and a second connecting terminal 602 are included at the connecting portion 105 in the body fluid container attaching portion 101 so as to be electrically connected to the first measuring electrode 501 and the second measuring electrode 502, respectively, when the body fluid container 500 is attached to the body fluid container attaching portion 101; a measuring voltage applying portion 603 is included, which applies a measurement voltage to the first measuring electrode 501 and the second measuring electrode 502; an electric signal measuring portion 604 is included, which measures an electric signal generated between the first measuring electrode 501 and the second measuring electrode 502; a third lead 605 and a fourth lead 606 are included, which electrically connect the first measuring electrode 501 to the measuring voltage applying portion 603 and the second measuring electrode 502 to the electric signal measuring portion 604, respectively; and a timer as the timer portion 403 is included, which measures time. Other than these, the body fluid measuring device 600 according to the present embodiment is the same in configuration as the body fluid sampling device 100 according to Embodiment 1. Therefore, same reference numbers are used for the same components, and explanations thereof are omitted.

The controller 118 of the body fluid measuring device 600 according to the present embodiment includes the memory. The memory prestores the calibration curve indicating the relationship between the concentration of glucose that is the specific substance of the measurement target and the electric signal measured by the electric signal measuring portion 604. The controller 118 of the body fluid measuring device 600 according to the present embodiment also functions as the calculating unit which measures the amount of the specific substance in the body fluid based on the electric signal measured by the electric signal measuring portion 604. This arithmetic function is realized by the calculating portion 118a included in the controller 118.

Next, the operation of the body fluid measuring device 600 according to the present embodiment will be explained.

Since the method for attaching the body fluid container 500 to the body fluid measuring device 600, the method for suctioning the body fluid into the body fluid container 500, the method for detecting the improper suctioning of the body fluid into the suctioning passage, and the method for discharging the body fluid from the body fluid container 500 are the same as those of Embodiment 1, explanations thereof are omitted in the following explanation.

In the body fluid measuring device 600 according to the present embodiment, when the introduction of the urine that is the body fluid sample into the space 302 of the body fluid container 500 is completed, the controller 118 causes the timer as the timer portion 403 to start timing. Simultaneously with the start of the timing, the controller 118 causes the measuring voltage applying portion 603 to apply the measurement voltage (for example, such a voltage that the potential of the first measuring electrode 501 is 0.5 V higher than that of the second measuring electrode 502) between the first measuring electrode 501 and the second measuring electrode 502.

Next, when the controller 118 determines based on a signal supplied from the timer portion 403 that a predetermined time (15 seconds for example) has passed since the completion of the introduction of the urine into the space 302 of the body fluid container 500, the controller 118 causes the electric signal measuring portion 604 to measure an electric signal of, for example, a current flowing between the first measuring electrode 501 and the second measuring electrode 502. The controller 118 reads out and refers to the calibration curve prestored in the memory, and thereby converts the electric signal measured by the electric signal measuring portion 604 into the concentration of glucose. The obtained concentration of glucose is displayed by the display portion 104 included in the body fluid measuring device 600. Since the display portion 104 displays the concentration of glucose, the user recognizes the completion of the measurement of the concentration of glucose.

As explained above, an electrochemical measurement of the body fluid can be carried out by using the body fluid measuring device 600 and the body fluid container 500 according to the present embodiment.

The present embodiment has explained a case of using the body fluid container 500 in which the body fluid introducing port 307 is formed on the side surface of the second member 309. However, the present embodiment is not limited to this. For example, instead of the body fluid introducing port 307 formed on the side surface of the second member 309, the body fluid introducing port may be formed on a tip end portion (lower end portion) of the body fluid container.

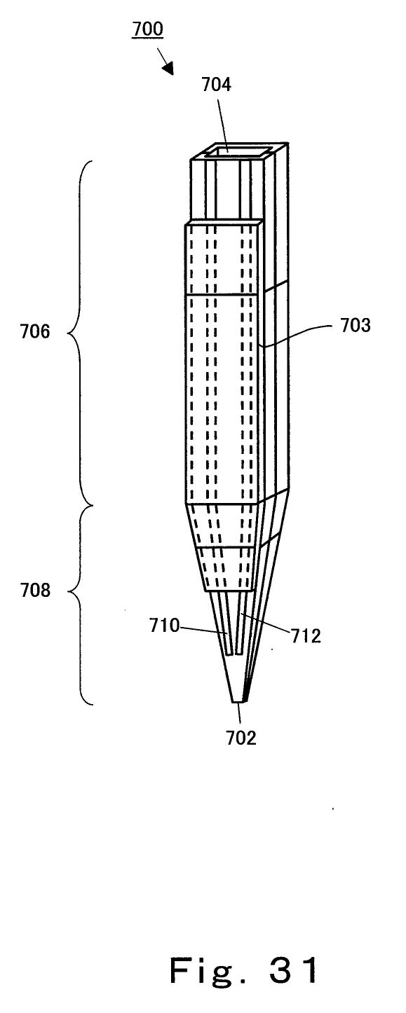

FIG. 31 is a perspective view schematically showing the configuration of an appearance of a modification example of the body fluid container used in Embodiment 6.

As shown in FIG. 31, a body fluid container 700 is made of transparent polystyrene. A first opening for obtaining the body fluid is formed at one end of the body fluid container 700, and a second opening for discharging the obtained body fluid toward the body fluid measuring device 600 is formed at the other end of the body fluid container 700. Moreover, a space is formed in the body fluid container 700. This space functions as the body fluid holding portion which holds the obtained body fluid. One opening end of the space functioning as the body fluid holding portion is a body fluid introducing port 702, and the other opening end is an opening 704.

More specifically, the body fluid container 700 includes a hollow quadrangular prism portion 706 and a hollow quadrangular pyramid portion 708. The opening 704 functioning as a suction port of the body fluid is formed at one end portion of the hollow quadrangular prism portion 706, and one end portion of the hollow quadrangular pyramid portion 708 is connected to (integral with) the other end portion of the hollow quadrangular prism portion 706. The body fluid introducing port 702 is formed at the other end portion of the hollow quadrangular pyramid portion 708.

As shown in FIG. 31, the body fluid container 700 includes a first measuring electrode 710 and a second measuring electrode 712. The first measuring electrode 710 and the second measuring electrode 712 extends on a side surface of the hollow quadrangular prism portion 706 from one end of the hollow quadrangular prism portion 706 to the other end thereof so as to be in parallel with each other, and extends on a side surface of the hollow quadrangular pyramid portion 708 from one end of the hollow quadrangular pyramid portion 708 to the other end thereof so as to be in a tapered shape. Moreover, predetermined portions of the first measuring electrode 710 and the second measuring electrode 712 are covered with a cover 703.

As above, by disposing the first measuring electrode 710 and the second measuring electrode 712 on a side surface of the body fluid container 700, it is possible to carry out an electrochemical measurement similar to the electrochemical measurement explained in the present embodiment. These electrodes do not have to be disposed in the case of not requiring the electrochemical measurement.

The above embodiments have explained a case where the ABS resin is used as the material of the cylinder 107 and the connecting portion 105. However, instead of this, for example, polypropylene resin may be used as the material of the cylinder 107 and the connecting portion 105.

Moreover, the present specification has explained a case where the shape of the cross section of each of the cylinder 107 and the connecting portion 105 is a cylindrical shape. However, the present invention is not limited to this. For example, the shape of the cross section of each of the cylinder 107 and the connecting portion 105 may be a tubular polygonal column, a tubular ellipsoid, a tubular truncated cone, a tubular truncated polygon, a tubular truncated ellipsoid, or a combination of these. Since the cylindrical shape and the tubular truncated cone among these shapes can most surely obtain the adhesion between the connecting portion 105 and the body fluid container 200, and the like, it is possible to surely suppress the inflow and leakage of gas from the connecting portion 105 which may occur at the time of suctioning. Therefore, it is most preferable that the shape of the cross section of each of the cylinder 107 and the connecting portion 105 be the cylindrical shape and the tubular truncated cone.

### INDUSTRIAL APPLICABILITY

The body fluid sampling device according to the present invention is industrially applicable to a field of using the body fluid for inspection and analysis, as a body fluid sampling device which can prevent the body fluid from being improperly suctioned into the body fluid sampling device and even if the body fluid is improperly suctioned, surely detect the improper suctioning of the body fluid. Moreover, the body fluid measuring device according to the present invention is industrially applicable as a preferable body fluid measuring device which includes the above characteristic body fluid sampling device and excels in operability and convenience.

1. A body fluid sampling device comprising:
a connecting portion to which a body fluid container is connected;
a suctioning portion which suctions a gas in the body fluid container connected to said connecting portion, to introduce a body fluid into the body fluid container;
a suctioning passage which causes an inside of said suctioning portion and an inside of the body fluid container connected to said connecting portion, to be communicated with each other;
a first electrode which is disposed to extend across said suctioning passage and has gas permeability;
a second electrode which is disposed to extend across said suctioning passage and be opposed to said first electrode, and has gas permeability; and
a detector which detects an electric signal generated between said first electrode and said second electrode.

2. The body fluid sampling device according to claim 1, further comprising a separator which is disposed between said first electrode and said second electrode and is able to be impregnated with the body fluid.

3. The body fluid sampling device according to claim 1, further comprising a filter which is disposed inside said suctioning passage and is able to suppress an intrusion of the body fluid.

4. The body fluid sampling device according to claim 3, wherein said filter is configured to contact at least one of said first electrode and said second electrode.

5. The body fluid sampling device according to claim 1, further comprising a surrounding member which integrally surrounds said first electrode and said second electrode.

6. The body fluid sampling device according to claim 1, further comprising a control portion which causes, based on the electric signal detected by said detector, said suctioning portion to stop suctioning of the gas.

7. A body fluid measuring device comprising:
the body fluid sampling device according to claim 1;
a light source which irradiates with light the body fluid introduced into the body fluid container connected to said connecting portion;
a photoreceiver which receives the light emitted from the body fluid container irradiated by said light source; and
a calculating unit which is used to measure the amount of a specific substance in the body fluid based on the light received by said photoreceiver.

8. A body fluid measuring device comprising:
the body fluid sampling device according to claim 1;
a pair of measuring electrodes disposed inside the body fluid container and a pair of connecting terminals which are electrically connectable to said pair of measuring electrodes; and
a calculating unit which is used to detect an electric signal generated between said pair of measuring electrodes via said pair of connecting terminals, and measure the amount of a specific substance in the body fluid based on the detected electric signal.
